SEQUENCE LISTING (46 sequences)

SEQ ID NO: 1 - DNA, 60 bp, Artificial Sequence
  Negative control sequence
gacttaaattcttcataactcgactacgagacctaatgtcggactaagttaaccaataaa

SEQ ID NO: 2 - DNA, 60 bp, Artificial Sequence
  Negative control sequence
tttattggttaacttagtccgacattaggtctcgtagtcgagttatgaagaatttaagtc

SEQ ID NO: 3 - DNA, 60 bp, Artificial Sequence
  Negative control sequence
tttgtaatctcgatacgcgtaagtttcgatcaggtaatttacatcgacatagacacccta

SEQ ID NO: 4 - DNA, 60 bp, Artificial Sequence
  Negative control sequence
tagggtgtctatgtcgatgtaaattacctgatcgaaacttacgcgtatcgagattacaaa

SEQ ID NO: 5 - DNA, 60 bp, Artificial Sequence
  Negative control sequence
cgataaaaagtcattgtatcgagtgataccgtaacctaccgttcgtagactattataaga

SEQ ID NO: 6 - DNA, 60 bp, Artificial Sequence
  Negative control sequence
tcttataatagtctacgaacggtaggttacggtatcactcgatacaatgactttttatcg

SEQ ID NO: 7 - DNA, 60 bp, Artificial Sequence
  Negative control sequence
tctcggtaaatagagtttcgtgcttatactagatgtagtctacgagatagacgctagatt

SEQ ID NO: 8 - DNA, 60 bp, Artificial Sequence
  Negative control sequence
aatctagcgtctatctcgtagactacatctagtataagcacgaaactctatttaccgaga

SEQ ID NO: 9 - DNA, 60 bp, Artificial Sequence
  Negative control sequence
aagtaacgtgagtagtatgatcatgttacgcgaggatcgttatcgagttacaataacata

SEQ ID NO: 10 - DNA, 60 bp, Artificial Sequence
  Negative control sequence
tatgttattgtaactcgataacgatcctcgcgtaacatgatcatactactcacgttactt

SEQ ID NO: 11 - DNA, 60 bp, Artificial Sequence
  Negative control sequence
tcgggtttacttgatatcaagcgcggttagaattgaatacgatgagacgaatttattaga

SEQ ID NO: 12 - DNA, 60 bp, Artificial Sequence
  Negative control sequence
tctaataaattcgtctcatcgtattcaattctaaccgcgcttgatatcaagtaaacccga

SEQ ID NO: 13 - DNA, 60 bp, Artificial Sequence
  Negative control sequence
atacgaatcttacgtagtttagtgtcgcttcactaaaaggctctatattcggatagtgca

SEQ ID NO: 14 - DNA, 60 bp, Artificial Sequence
  Negative control sequence
tgcactatccgaatatagagccttttagtgaagcgacactaaactacgtaagattcgtat

SEQ ID NO: 15 - DNA, 60 bp, Artificial Sequence
  Negative control sequence
ggctatcatagaaatgtagtcgaatcgtagcatactcgaattagatatctctatgctaag

SEQ ID NO: 16 - DNA, 60 bp, Artificial Sequence
  Negative control sequence
cttagcatagagatatctaattcgagtatgctacgattcgactacatttctatgatagcc

SEQ ID NO: 17 - DNA, 60 bp, Artificial Sequence
  Negative control sequence
caacgttgttatacgtcgttacctcaaaatgcgcgtaaaaacctgtgaactattataaag

SEQ ID NO: 18 - DNA, 60 bp, Artificial Sequence
  Negative control sequence
ctttataatagttcacaggtttttacgcgcattttgaggtaacgacgtataacaacgttg

SEQ ID NO: 19 - DNA, 60 bp, Artificial Sequence
  Negative control sequence
ttgaacttatgtaatctggtagtatcgagacaatcgttacagcgccatatgtaatgagaa

SEQ ID NO: 20 - DNA, 60 bp, Artificial Sequence
  Negative control sequence
ttctcattacatatggcgctgtaacgattgtctcgatactaccagattacataagttcaa

SEQ ID NO: 21 - DNA, 60 bp, Artificial Sequence
  Negative control sequence
tcgtgcagacttctacaacatcgagttctgcaacgtaataaccgtatgaataagactagt

SEQ ID NO: 22 - DNA, 60 bp, Artificial Sequence
  Negative control sequence
actagtcttattcatacggttattacgttgcagaactcgatgttgtagaagtctgcacga

SEQ ID NO: 23 - DNA, 60 bp, Artificial Sequence
  Negative control sequence
ctggtcttaatcgtcttgttaactaatacgggcatttacgagtcgatagacatataatca

SEQ ID NO: 24 - DNA, 60 bp, Artificial Sequence
  Negative control sequence
tgattatatgtctatcgactcgtaaatgcccgtattagttaacaagacgattaagaccag

SEQ ID NO: 25 - DNA, 60 bp, Artificial Sequence
  Negative control sequence
tgacaactagtttgcaatcgttataagtcgtattaacgcgaaattaacctgctaggaact

SEQ ID NO: 26 - DNA, 60 bp, Artificial Sequence
  Negative control sequence
agttcctagcaggttaatttcgcgttaatacgacttataacgattgcaaactagttgtca

SEQ ID NO: 27 - DNA, 60 bp, Artificial Sequence
  Negative control sequence
attagaactactataaatccggcgagattctatggcgcataacatgatagacagaacatt

SEQ ID NO: 28 - DNA, 60 bp, Artificial Sequence
  Negative control sequence
aatgttctgtctatcatgttatgcgccatagaatctcgccggatttatagtagttctaat

SEQ ID NO: 29 - DNA, 60 bp, Artificial Sequence
  Negative control sequence
gttaccgtttgaataataacggacggataaccctttgatacatcccaacgtataataagg

SEQ ID NO: 30 - DNA, 60 bp, Artificial Sequence
  Negative control sequence
ccttattatacgttgggatgtatcaaagggttatccgtccgttattattcaaacggtaac

SEQ ID NO: 31 - DNA, 60 bp, Artificial Sequence
  Negative control sequence
gtagagtatattgctttaatacgaccccgataagcacgatcgtattagacatagatgata

SEQ ID NO: 32 - DNA, 60 bp, Artificial Sequence
  Negative control sequence
tatcatctatgtctaatacgatcgtgcttatcggggtcgtattaaagcaatatactctac

SEQ ID NO: 33 - DNA, 60 bp, Artificial Sequence
  Negative control sequence
ataattcgttgactatagcacatttcgatcctcgttatgataccaatgaacggaagtctt

SEQ ID NO: 34 - DNA, 60 bp, Artificial Sequence
  Negative control sequence
aagacttccgttcattggtatcataacgaggatcgaaatgtgctatagtcaacgaattat

SEQ ID NO: 35 - DNA, 60 bp, Artificial Sequence
  Negative control sequence
cagatcgatcggtttatatgcgatttaacgccgctttcatcctaaagcgcaaattttaca

SEQ ID NO: 36 - DNA, 60 bp, Artificial Sequence
  Negative control sequence
tgtaaaatttgcgctttaggatgaaagcggcgttaaatcgcatataaaccgatcgatctg

SEQ ID NO: 37 - DNA, 60 bp, Artificial Sequence
  Negative control sequence
tacgtcaattcgtgatatgcctttcgattatcataccgaagagtcctttagtaagtttag

SEQ ID NO: 38 - DNA, 60 bp, Artificial Sequence
  Negative control sequence
ctaaacttactaaaggactcttcggtatgataatcgaaaggcatatcacgaattgacgta

SEQ ID NO: 39 - DNA, 60 bp, Artificial Sequence
  Negative control sequence
gaaactagtgaaacagagttcgctaagcgtctaaactcgagtttttacgaactaatacaa

SEQ ID NO: 40 - DNA, 60 bp, Artificial Sequence
  Negative control sequence
ttgtattagttcgtaaaaactcgagtttagacgcttagcgaactctgtttcactagtttc

SEQ ID NO: 41 - DNA, 60 bp, Artificial Sequence
  Negative control sequence
ggtattgttcttatattcatcgtgaccagtaaccaattgatatcggatttcggtttacag

SEQ ID NO: 42 - DNA, 60 bp, Artificial Sequence
  Negative control sequence
ctgtaaaccgaaatccgatatcaattggttactggtcacgatgaatataagaacaatacc

SEQ ID NO: 43 - DNA, 60 bp, Artificial Sequence
  Negative control sequence
ctatttctcgaaaccgttaaatcgaaatgttatgtccgctaatcgaaccactaatcgttt

SEQ ID NO: 44 - DNA, 60 bp, Artificial Sequence
  Negative control sequence
aaacgattagtggttcgattagcggacataacatttcgatttaacggtttcgagaaatag

SEQ ID NO: 45 - DNA, 19 bp, Artificial Sequence
  Forward PCR primer for lambda phage
ctggatgtcgctccacaaa

SEQ ID NO: 46 - DNA, 24 bp, Artificial Sequence
  Reverse PCR primer for lambda phage
ttgatcgccagatagtggtgcttc

(19) United States
(12) Patent Application Publication
(10) Pub. No.: US 2008/0102452 A1
(43) Pub. Date: May 1, 2008

(54) Control nucleic acid constructs for use in analysis of methylation status

(76) Inventors: Douglas N. Roberts, Campbell, CA (US); Stephen B. Milligan, Los Altos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/590,711

(22) Filed: Oct. 31, 2006

(51) Int. Cl. C12Q 1/68; C07H 21/04

(52) U.S. Cl. 435/6; 536/24.3

(57) ABSTRACT

In some embodiments, control nucleic acid constructs useful as spiking reagents are provided which comprise a nucleic acid vector having an insert comprising a control nucleic acid molecule. In some embodiments, the insert contains at least one methyltransferase recognition site, such as a CpG dinucleotide. In some embodiments, the insert has a sequence complementary to a negative control probe of a microarray. Methods and kits for using the control nucleic acid constructs as spiking reagents in methylation analysis are disclosed.

### BACKGROUND

The human genome is estimated to contain 50×10⁶ CpG dinucleotides, the predominant sequence recognition motif for mammalian DNA methyltransferases. Clusters of CpGs, or “CpG islands”, are present in the promoter or intronic regions of approximately 40% of mammalian genes (Larsen et al. (1992) Genomics 13:1095-1107). Methylation of cytosine residues contained within CpG islands (i.e., “CpG island methylation”) has generally been correlated with reduced gene expression, and is thought to play a fundamental role in many mammalian processes, including embryonic development, X-inactivation, genomic imprinting, regulation of gene expression, and host defense against parasitic sequences, as well as abnormal processes such as carcinogenesis, fragile site expression, and cytosine to thymine transition mutations. In addition alterations in methylation levels of CpGs occur under different physiologic and pathologic conditions. Accordingly, CpG methylation is an area of intense interest to the scientific community.

Many CpG sites within a genome are found in a methylated state, and some CpG sites occur near coding regions within the genome. Such methylation has been linked to gene expression. Additionally, alterations in DNA methylation within a genome often are a manifestation of genomic instability, which may be a characteristic sign of a tumor. Thus, techniques for determining the methylation of DNA find use in many different applications.

Various methods exist for the isolation and detection of specific patterns of DNA methylation, including gels, capillary systems, PCR and arrays. Chemical arrays have gained prominence in biological research and serve as valuable diagnostic tools in the healthcare industry. A fundamental principle upon which array assays are based is that of specific recognition. Probe molecules affixed to the array can specifically recognize and bind target molecules in a sample, either by sequence-mediated binding affinities, binding affinities based on conformational or topological properties of probe and target molecules, or binding affinities based on spatial distribution of electrical charge on the surfaces of target and probe molecules.

An array generally includes a substrate upon which a regular pattern of features is prepared by various manufacturing processes. The array typically has a grid-like two-dimensional pattern of features. For nucleic acid arrays, each feature of the array contains a large number of oligonucleotides covalently bound to the surface of the feature. These bound oligonucleotides are known as probes. In general, chemically distinct probes are bound to the different features of an array, so that each feature corresponds to a particular known nucleotide sequence.

Once an array has been prepared, the array can be exposed to a sample solution containing target molecules (such as DNA or RNA) labeled with fluorophores, chemiluminescent compounds, or radioactive atoms. The labeled target molecules then hybridize to the complementary probe molecules on the surface of the array. Targets, such as labeled DNA molecules that are not complementary to any of the probes bound to array surface do not hybridize as readily and tend to remain in solution. The sample solution is then rinsed from the surface of the array, washing away any unbound labeled molecules. Finally, the bound labeled molecules are detected via optical or radiometric scanning.

Scanning of an array by an optical scanning device or radiometric scanning device generally produces a scanned image comprising a plurality of pixels corresponding to features on the array, with each pixel having a corresponding signal intensity. Typically, an array-data-processing program then manipulates these signal intensities and produces experimental or diagnostic results.

There is a need for exogenous nucleic acid controls (“spikes”) for analysis of DNA methylation using various analytical systems, including microarrays. Variations in sample preparation, hybridization conditions, and array quality can influence the analysis. The use of quality-assured control polynucleotides during sample preparation and analysis can enhance the ability to normalize data and to compare experiments, as well as to monitor each step of the assay.

### SUMMARY

In some aspects, control nucleic acid constructs useful as spiking reagents in DNA methylation analysis, are provided. In some embodiments, a control nucleic acid construct comprises a nucleic acid vector comprising one or more inserted sequences. In some embodiments, an insert comprises a sequence complementary to a negative control sequence of a microarray. In some embodiments, the insert comprises a methyltransferase recognition site. In some embodiments, the insert comprises a methylated methyltransferase recognition site. Non-limiting examples of a methyltransferase recognition site include CpG, CpA, CpT, CpNpG, ApG, GpG, CCGG, GGCC, and TCGA. Non-limiting examples of a methylation site include 5-methyl cytidine, 6-methyl adenosine, and 7-methyl guanosine. The length of a control nucleic acid construct can range in size from about 1 kilobases (kb) to about 100 kb. The length of an inserted sequence can be in the range of about 5 to about 1000 bases. In some embodiments, an insert has a length of 60 bases.

The vector can be a viral nucleic acid vector, a non-limiting example of which is lambda phage gt11. In some embodiments, a control nucleic acid molecule comprising a sequence complementary to a negative control sequence of a microarray is inserted into a restriction site (such as, for example, an EcoR1 restriction site) in the vector. In some embodiments, a spiking reagent comprises a PCR amplification product of the control nucleic acid construct wherein the amplification product comprises the inserted control nucleic acid molecule. In some embodiments, an additional insert flanking the control nucleic acid molecule is provided, and wherein the additional insert can comprise one or more methyltransferase recognition site. In some embodiments, the additional insert can comprise a methylated methyltransferase recognition site. In some embodiments, the additional insert can comprise one or more methylated CpG dinucleotides. In some embodiments, the vector sequence (independent of any insert sequence(s)) has been modified to deplete the vector sequence of methyltransferase recognition site(s) (such as, for example, CpG dinucleotides). Also provided, are mixtures of control nucleic acid constructs, or amplification products thereof, for use as spiking reagents. Also provided, are compositions comprising said control nucleic acid constructs, or amplification products thereof, having various degrees of saturation of methylation, for example, ranging from 0% to 100% saturation of methylation.

Provided are methods for preparing control nucleic acid constructs as described herein. In some embodiments, the methods comprise conventional oligonucleotide synthesis procedures. In some embodiments, the methods can comprise conventional cloning procedures.

In some aspects, there are provided methods for assessing methylation status of a sample. In some embodiments, the methods comprise: a) adding a control nucleic acid construct to said sample, said construct comprising a nucleic acid vector comprising an insert comprising a sequence complementary to a negative control sequence, wherein said insert comprises a methylation site, b) enriching said sample for nucleic acids comprising a methylated methylation site, and c) detecting nucleic acids obtained in step (b) to assess the methylation status of said sample. In some embodiments, the enrichment step can comprise immunoprecipitation of nucleic acids comprising a methylated methylation site. The methods can include fragmentation steps, amplification steps, and labeling steps. The detecting can comprise various methods using PCR, blots or arrays.

In some embodiments, there are provided methods for detection of changes in nucleic acid methylation in a patient over time comprising: (i) obtaining a tissue specimen from the patient at a time point; (ii) repeating step (i) for at least one further time point; (iii) extracting nucleic acid from each tissue specimen to provide a sample of nucleic acid for each time point, and (iv) carrying out a method for assessing methylation status as described herein on each nucleic acid sample for each time point to characterize whether, and/or to what extent, the nucleic acid sequence is methylated.

Compositions and kits comprising spike-in reagents are encompassed within the scope of the disclosure herein, as are arrays that comprise probes complementary to the spike-in reagents.

The instant control nucleic acid constructs (or amplification products thereof can be added to a sample of target nucleic acids being analyzed for methylation status to allow a user to assess any degradation in the overall performance of the analysis, including, but not limited to, signal-to-noise, dynamic range, linearity of response, and background. Spike-in controls for the process of isolation and analysis of methylated DNA, as described herein, can provide increased confidence in the isolation and detection procedure.

Additional objects, advantages, and features of the present disclosure will become apparent from the following description taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments can be more completely understood in connection with the following drawings, in which:

FIG. 1 schematically illustrates some embodiments of a control nucleic acid construct.

FIG. 2 schematically illustrates some embodiments of a control nucleic acid construct.

FIG. 3 illustrates a schematic diagram of a system for manufacturing arrays.

FIG. 4 illustrates some examples of a general purpose computing system.

FIG. 5 shows operations performed in some embodiments.

FIG. 6 shows operations of similarity screening performed in some embodiments.

FIG. 7. illustrates a scatter plot of data obtained from a hybridization experiment showing data from negative control probes and also showing data from genomic probes.

FIG. 8. illustrates the plot of FIG. 7 but without the data from genomic probes.

### DETAILED DESCRIPTION

The present disclosure generally relates to the determination of the state of one or more locations within a nucleic acid and, in particular, to the determination of the methylation state of one or more methylation sites within a nucleic acid such as DNA.

DNA is a molecule that is present within all living cells. DNA encodes genetic instructions which tell the cell what to do. By “examining” the instructions, the cell can produce certain proteins or molecules, or perform various activities. DNA itself is a long, linear molecule where the genetic information is encoded using any one of four possible “bases,” or molecular units, in each position along the DNA. This is roughly analogous to “beads on a string,” where a string may have a large number of beads on it, encoding various types of information, although each bead along the string can only be of one of four different colors.

In some cases, however, the cell may “methylate” a base on the DNA, which is a chemical reaction that subtly alters the base in a way that the cell can later recognize it. This may be performed for various reasons, such as to indicate that a particular piece of information is no longer important to the cell. The cell may also “demethylate” the base in some cases, e.g., to indicate that the information is again important to the cell. Extending the above “beads on a string” analogy, this would be akin to marking a bead with a piece of tape, which could later be removed, if necessary.

Scientists who study cells are interested in observing which bases along a given piece of DNA have been methylated. This has important implications in fields such as cancer research or research into hereditary diseases. However, as DNA is small and difficult to work with, scientists are interested in techniques for discovering which bases along the DNA have been methylated. Disclosed herein are novel compositions and techniques useful in the determination of methylation status.

Before describing the present disclosure in detail, it is to be understood that this disclosure is not limited to specific compositions, method steps, or equipment, as such can vary. It is also to be understood that the terminology used herein is for the purpose of describing particular embodiments only, and is not intended to be limiting. Methods recited herein can be carried out in any order of the recited events that is logically possible, as well as the recited order of events. Furthermore, where a range of values is provided, it is understood that every intervening value, between the upper and lower limit of that range and any other stated or intervening value in that stated range is encompassed within the present disclosure. Also, it is contemplated that any optional feature of the disclosed variations described can be set forth and claimed independently, or in combination with any one or more of the features described herein.

Unless defined otherwise below, all technical and scientific terms used herein have the same meaning as commonly understood by one of ordinary skill in the art to which this disclosure belongs. Still, certain elements are defined herein for the sake of clarity.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” can include more than one biopolymer.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### Definitions

The following definitions are provided for specific terms that are used in the following written description.

A “biopolymer” is a polymer of one or more types of repeating units. Biopolymers are typically found in biological systems and can include polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “nucleotide” refers to a sub-unit of a nucleic acid and has a phosphate group, a 5-carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 200 nucleotides in length, while a “polynucleotide” or “nucleic acid” includes a nucleotide multimer having any number of nucleotides.

The term “base composition properties” shall refer to properties of a sequence related to base composition. By way of example, while not limiting the term, base composition properties can include the percentage of A, C, T, and G sequences within a given probe sequence.

The term “primary structural features” as used herein shall refer to structural features of a sequence related the contiguous positioning of bases in the sequence. While not limiting the term, an example of a primary structural feature is a homopolymeric run.

The term “homopolymeric run” as used herein shall refer to a portion of a base sequence wherein a given base is repeated more than once. By way of example, a sequence contains the contiguous bases “TTTTT” would be considered to have a homopolymeric run.

The term “secondary structural features” as used herein shall refer to structural features (predicted or empirical) of a sequence caused by the interaction between both contiguous and non-contiguous bases in the sequence. While not limiting the term, an example of a secondary structural feature is a hairpin loop structure.

As used herein, the term “thermodynamic characteristics” shall refer to characteristics of a sequence described in thermodynamic terms. By way of example, while not limiting the term, thermodynamic characteristics of a given sequence can include the Gibbs free energy of hybridization of that sequence with another sequence. As a further example, while not limiting the term, thermodynamic characteristics of a given sequence can include the melting temperature (Tm) of the sequence.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties can be covalently or non-covalently bound to the surface in the region. For example, each region can extend into a third dimension in the case where the substrate is porous while not having any substantial third dimension measurement (thickness) in the case where the substrate is non-porous. An array can contain more than ten, more than one hundred, more than one thousand more than ten thousand features, or even more than one hundred thousand features, in an area of less than 20 cm² or even less than 10 cm². For example, features can have widths (that is, diameter, for a round spot) in the range of from about 10 μm to about 1.0 cm. In other embodiments each feature can have a width in the range of about 1.0 μm to about 1.0 mm, such as from about 5.0 μm to about 500 μm, and including from about 10 μm to about 200 μm. Non-round features can have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features can account for at least 5%, 10%, or 20% of the total number of features). Interfeature areas can be present which do not carry any polynucleotide. Such interfeature areas typically can be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature can incidentally detect non-targets of that feature). The target for which each feature is specific is, in representative embodiments, known. An array feature is generally homogenous in composition and concentration and the features can be separated by intervening spaces (although arrays without such separation can be fabricated).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In some embodiments, the collections of oligonucleotide elements employed herein are present on a surface of the same planar support, e.g., in the form of an array. It should be understood that the terms “probe” and “target” are relative terms and that a molecule considered as a probe in certain assays can function as a target in other assays.

An “unstructured nucleic acid” or “UNA” for short (see, e.g., US Patent Application Publication 20050233340) is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

With arrays that are read by detecting fluorescence, the substrate can be of a material that emits low fluorescence upon illumination with the excitation light. Additionally, the substrate can be relatively transparent to reduce the absorption of the incident illuminating laser light and subsequent heating if the focused laser beam travels too slowly over a region.

In some embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO₄, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unbound nucleic acids can include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and can also be employed, as appropriate. The term “highly stringent hybridization conditions” as used herein refers to conditions that are compatible to produce complexes between complementary binding members, i.e., between immobilized probes and complementary sample nucleic acids, but which do not result in any substantial complex formation between non-complementary nucleic acids (e.g., any complex formation which cannot be detected by normalizing against background signals to interfeature areas and/or control regions on the array).

Stringent hybridization conditions can also include a “prehybridization” of aqueous phase nucleic acids with complexity-reducing nucleic acids to suppress repetitive sequences and reduce the complexity of the sample prior to hybridization. For example, certain stringent hybridization conditions include, prior to any hybridization to surface-bound polynucleotides, hybridization with Cot-1 DNA, or the like.

Additional hybridization methods are described in Kallioniemi et al. (1992) Science 258:818-821 and WO 93/18186. Several guides to general techniques are available, e.g., Tijssen, Hybridization with Nucleic Acid Probes, Parts I and II (Elsevier, Amsterdam, 1993). For descriptions of techniques suitable for in situ hybridizations see, Gall et al. (1981) Meth. Enzymol. 21:470-480 and Angerer et al., In Genetic Engineering: Principles and Methods, Setlow and Hollaender, Eds. Vol 7, pgs 43-65 (Plenum Press, New York, 1985). See also U.S. Pat. Nos. 6,335,167; 6,197,501; 5,830,645; and 5,665,549; the disclosures of which are herein incorporated by reference.

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

In some embodiments, a target nucleic acid to be probed may be any nucleic acid which includes, or is suspected to include, a methylation site. The nucleic acid may be, for example, DNA or RNA, and the nucleic acid may arise from any suitable source, for example, genomic DNA (which may be whole or fragmented, e.g., enzymatically and/or mechanically), mitochondrial DNA, cDNA, synthetic DNA, or the like. The target nucleic acid may have any suitable length. For example, the nucleic acid may have a length of at least about 10 nucleotides, at least about 25 nucleotides, at least about 40 nucleotides, at least about 50 nucleotides, at least about 75 nucleotides, at least about 100 nucleotides, at least about 300 nucleotides, at least about 1,000 nucleotides, at least about 10,000 nucleotides, at least about 100,000 nucleotides, etc. In some cases, for example, with genomic DNA, the nucleic acid may optionally first be cleaved, for instance, using chemicals or restriction endonucleases known to those of ordinary skill in the art, prior to determining methylation of the methylation site.

A “methylation site,” as used herein, is given its ordinary definition as used in the art, i.e., a base within a nucleic acid in which a hydrogen atom of the base can be enzymatically replaced by a methyl (—CH₃) group. Examples of methylated nucleosides include methylated cytidine (e.g., 5-methyl cytidine), methylated adenosine (e.g., 6-methyl adenosine) and methylated guanosine (7-methyl guanosine). The most common methylation site is the cytosine base of a “CpG” sequence within DNA, i.e., a cytosine followed by a guanine within the DNA strand (the “p” in the abbreviation “CpG” stands for the intervening phosphate between the two bases). Typically, the hydrogen in the “5” position of the cytosine is replaced by a methyl, forming 5-methylcytosine. CpG sequences have been linked to gene regulation, as well as changes or errors in gene expression, for example, in epigenetics or in cancer cells. In a nucleic acid duplex (two antiparallel strands associated at substantially complementary regions), if only one strand is methylated at a methylation site, the duplex is “hemi-methylated.” If both strands are methylated at the methylation site, the duplex is “fully methylated.” For purposes of simplifying the description herein and not by way of limitation, the methylation of cytosine in a CpG dinucleotide will be primarily described herein, it being understood that other methylation sites are intended to be included within the scope of this disclosure.

CpG sequences within genomic DNA are often not randomly distributed, but are instead typically found in high concentrations in certain portions of the DNA, known as “CpG islands.” Some of the CpG islands have been linked to promoter sites. The CpG islands within DNA are generally rich in cytosine and guanine, some of which are located next to each other to form CpG pairs which are susceptible to methylation, as described above. However, in a CpG island; the cytosine and guanine residues do not necessarily have to occur at the same frequency or always be in a “CpG” repeat sequence. Those of ordinary skill in the art will be able to identify CpG islands within DNA. For instance, the CpG island may include at least about 50 nucleotides, and in some cases, the CpG island may include at least about 100 nucleotides or at least about 200 nucleotides. Within the CpG island, the frequency of appearance of cytosine and guanine may be significantly greater than chance (i.e., significantly greater than 25% for each, or 50% for both), and the frequency of each may be the same or different. For instance, within the CpG island, the combined frequency of cytosine and guanine may be at least about 60%, at least about 65%, at least about 70%, or at least about 75%, and cytosine and guanine may appear in the same or different percentages. As a non-limiting example, a CpG island may be identified as a region having between about 200 nucleotides and about 800 nucleotides, with a combined frequency of appearance of both cytosine and guanine greater than about 60% or about 65%.

A CpG island is defined as any discrete region of a genome that contains a CpG that is, or is predicted to be, a target for a cellular methyltransferase. CpG islands may be high-density CpG islands, such as those defined by Gardiner-Garden and Frommer (1987) J. Mol. Biol. 196:261-282, i.e., any stretch of DNA that is at least 200 bp in length that has a C+G content of at least 50% and an observed CpG/expected CpG ratio of greater than or equal to 0.60. CpG islands may also be low-density CpG islands, containing CpG dinucleotides that occur at a lower density in a given region. The methylation status of these low density CpG islands varies under different physiologic and pathologic conditions, including aging and cancer (Toyota and Issa (1999) Seminars in Cancer Biology 9:349-357). In general, CpG islands are generally found proximal to (i.e., within 1 kb, 3 kb, or about 5 kb of) the transcriptional start sites of eukaryotic genes. It has been estimated that there are approximately 45,000 CpG islands in the human genome and 37,000 CpG islands in the mouse genome (Antequera et al. (1993) Proc. Natl. Acad. Sci. 90:11995-11999).

A detailed discussion of CpG islands, methods for their identification, and many examples of CpG islands in human chromosomes is found in a variety of publications, including: Larsen, et al. (1992) Genomics 13:1095-1107; Takai et al. (2002) Proc. Natl. Acad. Sci. 99:3740-3745; Antequera et al. (1993); and Ioshikhes et al. (2000) Nat. Genet. 26:61-63.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spacially distinct. In other words, a mixture is not addressable. To be specific, an array of surface-bound polynucleotides, as is commonly known in the art and described below, is not a mixture of surface-bound polynucleotides because the species of surface-bound polynucleotides are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

If a subject CpG oligonucleotide “corresponds to” or is “for” a certain CpG island, the oligonucleotide usually base pairs with, i.e., specifically hybridizes to, that CpG island. A CpG oligonucleotide for a particular CpG island and the particular CpG island, or complement thereof, usually contain at least one region of contiguous nucleotides that is identical in sequence (with the exception of any modified nucleotides).

As used herein, a “biologically occurring sequence” refers to a sequence in a biological sample of target nucleic acids, e.g., such as a sequence from a biological organism, cell, tissue type, etc., being evaluated by hybridization to a collection of probe molecules which are designed to detect one or more sequences in the biological sample (e.g., by specifically hybridizing to the sequence under stringent conditions).

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that can be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell can contain variable numbers of each chromosome in addition to deletions, rearrangements and amplification of any subchromosomal region or DNA sequence. In some embodiments, a “genome” refers to nuclear nucleic acids, excluding mitochondrial nucleic acids; however, in some embodiments, the term does not exclude mitochondrial nucleic acids. In some embodiments, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols.

The term “sample” as used herein relates to a material or mixture of materials, containing one or more components of interest. Samples include, but are not limited to, samples obtained from an organism or from the environment (e.g., a soil sample, water sample, etc.) and can be directly obtained from a source (e.g., such as a biopsy or from a tumor) or indirectly obtained e.g., after culturing and/or one or more processing steps. In some embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

As used herein, a “test nucleic acid sample” or “test nucleic acids” refer to nucleic acids comprising sequences whose degree of methylation is being assayed. Similarly, “test genomic acids” or a “test genomic sample” refers to genomic nucleic acids comprising sequences whose degree of methylation or sequence identity is being assayed.

If a surface-bound polynucleotide or probe “corresponds to” a chromosomal region, the polynucleotide usually contains a sequence of nucleic acids that is unique to that chromosomal region. Accordingly, a surface-bound polynucleotide that corresponds to a particular chromosomal region usually specifically hybridizes to a labeled nucleic acid made from that chromosomal region, relative to labeled nucleic acids made from other chromosomal regions.

In some embodiments, an array comprises probe sequences for scanning an entire chromosome arm, wherein probes are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In some embodiments, an array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes can also vary, and can be uniform, such that the spacing is substantially the same between sampled regions, or non-uniform, as desired. An assay performed at low resolution on one array, e.g., comprising probe targets separated by larger distances, can be repeated at higher resolution on another array, e.g., comprising probe targets separated by smaller distances.

In some embodiments, in constructing an array, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions can include regulatory sequences, e.g., promoters, enhancers, untranslated but transcribed regions, introns, origins of replication, telomeres, etc. In some embodiments, one can have at least some of the probes directed to non-coding regions and others directed to coding regions. In some embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In some embodiments, at least 5% of the polynucleotide probes on the solid support hybridize to regulatory regions of a nucleotide sample of interest while other embodiments can have at least 30% of the polynucleotide probes on the solid support hybridize to exonic regions of a nucleotide sample of interest. In some embodiments, at least 50% of the polynucleotide probes on the solid support hybridize to intergenic regions (e.g., non-coding regions which exclude introns and untranslated regions, i.e., comprise non-transcribed sequences) of a nucleotide sample of interest.

In some embodiments, probes on an array represent a random selection of genomic sequences (e.g., both coding and noncoding). However, in some embodiments, particular regions of the genome are selected for representation on an array, e.g., such as genes belonging to particular pathways of interest or whose expression and/or copy number are associated with particular physiological responses of interest (e.g., disease, such a cancer, drug resistance, toxological responses and the like). In some embodiments, where particular genes are identified as being of interest, intergenic regions proximal to those genes are included on an array along with, optionally, all or portions of the coding sequence corresponding to the genes. In some embodiments, at least about 100 bp, 500 bp, 1,000 bp, 5,000 bp, 10,000 bp or even 100,000 bp of genomic DNA upstream of a transcriptional start site is represented on an array in discrete or overlapping sequence probes. In some embodiments, at least one probe sequence comprises a motif sequence to which a protein of interest (e.g., such as a transcription factor) is known or suspected to bind.

In some embodiments, repetitive sequences are excluded as probes on an array. However, in some embodiments, repetitive sequences are included.

The choice of nucleic acids to use as probes can be influenced by prior knowledge of the association of a particular chromosome or chromosomal region with certain disease conditions. International Application WO 93/18186 provides a list of exemplary chromosomal abnormalities and associated diseases, which are described in the scientific literature. Whole genome screening to identify new regions subject to frequent changes in methylation can be performed using the methods presently disclosed.

In some embodiments, previously identified regions from a particular chromosomal region of interest are used as probes. In some embodiments, an array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density can be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

“Themed” arrays can be fabricated, for example, as arrays including probes associated with specific types of cancer (e.g., breast cancer, prostate cancer and the like). The selection of such arrays can be based on patient information such as familial inheritance of particular genetic abnormalities. In some embodiments, an array for scanning an entire genome is first contacted with a sample and then a higher-resolution array is selected based on the results of such scanning. Themed arrays can be fabricated for use in methylation assays, for example, to detect methylation of genes involved in selected pathways of interest, or genes associated with particular diseases of interest.

In some embodiments, a plurality of probes on an array are selected to have a duplex Tₘ within a predetermined range. For example, in some embodiments, at least about 50% of the probes have a duplex Tₘ within a temperature range of about 70° C. to about 100° C. In some embodiments, at least about 50% of the probes have a duplex Tₘ within a temperature range of about 75° C. to about 85° C. In some embodiments, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In some embodiments, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in some embodiments, have a nucleotide length in the range of at least 30 nucleotides to 200 nucleotides, or in the range of at least about 30 to about 150 nucleotides. In some embodiments, at least about 50% of the polynucleotide probes on the solid support have the same nucleotide length, and that length can be about 60 nucleotides.

In some embodiments, probes on an array comprise at least coding sequences.

In some embodiments, probes represent sequences from an organism such as Drosophila melanogaster, Caenorhabditis elegans, yeast, zebrafish, a mouse, a rat, a domestic animal, a companion animal, a primate, a human, etc. In some embodiments, probes representing sequences from different organisms are provided on a single substrate, e.g., on a plurality of different arrays.

Methods to fabricate arrays are described in detail in U.S. Pat. Nos. 6,242,266; 6,232,072; 6,180,351; 6,171,797 and 6,323,043. Drop deposition methods can be used for fabrication, as previously described herein. Also, instead of drop deposition methods, photolithographic array fabrication methods can be used. Interfeature areas need not be present particularly when an array is made by photolithographic methods as described in those patents.

Following receipt by a user, an array can be exposed to a sample and then read. Reading of an array can be accomplished by illuminating the array and reading the location and intensity of resulting fluorescence at multiple regions on each feature of the array. For example, a scanner can be used for this purpose, such as the AGILENT MICROARRAY SCANNER manufactured by Agilent Technologies (Santa Clara, Calif.) or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which can be directly inputted to a feature extraction system for direct processing and/or saved in a computer storage device for subsequent processing. However, arrays can be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

It should also be noted that, as used in this specification and the appended claims, the term “configured” describes a system, apparatus, or other structure that is constructed or configured to perform a particular task or adopt a particular configuration to. The phrase “configured” can be used interchangeably with other similar phrases such as arranged and configured, constructed and arranged, adapted, constructed, manufactured and arranged, and the like.

As used herein, the term “determining” generally refers to the analysis of a species, for example, quantitatively or qualitatively, and/or the detection of the presence or absence of the species. “Determining” may also refer to the analysis of an interaction between two or more species, for example, quantitatively or qualitatively, and/or by detecting the presence or absence of the interaction. In addition, the terms “determining,” “measuring,” “evaluating,” “assessing,” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The practice of the present methods can employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Some embodiments of suitable techniques can be had by reference to the examples hereinbelow. However, other equivalent conventional procedures can, of course, also be used. Such conventional techniques and descriptions can be found in standard laboratory manuals such as Genome Analysis: A Laboratory Manual Series (Vols. I-IV); Using Antibodies: A Laboratory Manual; Cells: A Laboratory Manual; PCR Primer: A Laboratory Manual; and Molecular Cloning: A Laboratory Manual (all from Cold Spring Harbor Laboratory Press), Stryer, “Biochemistry” (WH Freeman); Gait, “Oligonucleotide Synthesis: A Practical Approach” 1984, IRL Press, London; Freifelder, D “Molecular Biology” 2^{nd} edition, Jones & Bartlett (1987); Ausubel et al. eds., “Current Protocols in Molecular Biology”, chapters 1-3, John Wiley (1994) all of which are herein incorporated in their entirety by reference for all purposes.

Control Nucleic Acid Constructs

Control nucleic acid constructs as described herein can be used as a reference to spike samples of nucleic acids, such as a test sample or a reference sample, prior to processing and analysis steps. A “spike” or “spiking reagent” refers to a reagent having a known composition which can be added to a sample at a known concentration and which acts as an internal control during preparation and analysis to monitor method performance.

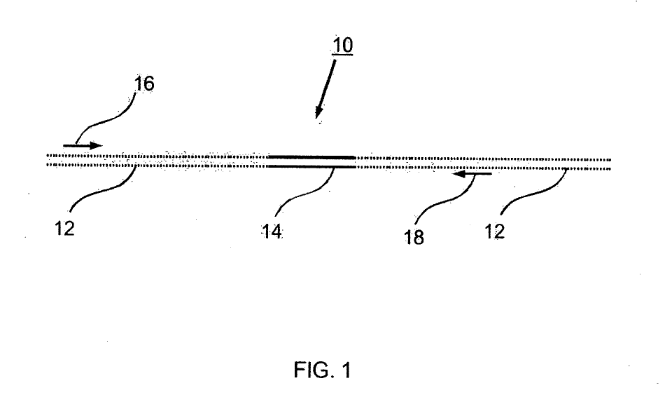

In some embodiments, a nucleic construct 10 comprises a vector 12 having a control nucleic acid molecule 14 inserted therein (FIG. 1). In some embodiments, only a single control nucleic acid molecule is inserted. In some embodiments, more than one control nucleic acid molecule is inserted. The sequence of the control nucleic acid molecule as disclosed herein can be complementary to a negative control probe, as described hereinbelow and in U.S. patent application Ser. No. 11/292,588, the disclosure of which is incorporated by reference herein.

The length of insert 14 can be selected as needed and will depend upon the length of the complementary negative control probe under consideration. In some embodiments, the length of insert 14 can be in the range of 20 to 100 nucleotides, 10 to 200 nucleotides, or 10 to 500 nucleotides, for example. In some embodiments, the length of insert 14 is 60 nucleotides. In some embodiments, the length of insert 14 is 200 nucleotides. Non-limiting examples of control nucleic acid molecules include SEQ ID NOs: 1-44 as shown in Table 1.

TABLE 1

| | | |
|---|---|---|
| SEQ ID | Orien- | |
| NO: | tation | Control nucleic acid molecule |

| | | |
|---|---|---|
| 1 | 5′-3′ | GACTTAAATTCTTCATAACTCGACTACGAGACCTAATGTCGGACTAAGTTAACCAATAAA |
| 2 | 3′-5′ | CTGAATTTAAGAAGTATTGAGCTGATGCTCTGGATTACAGCCTGATTCAATTGGTTATTT |
| 3 | 5′-3′ | TTTGTAATCTCGATACGCGTAAGTTTCGATCAGGTAATTTACATCGACATAGACACCCTA |
| 4 | 3′-5′ | AAACATTAGAGCTATGCGCATTCAAAGCTAGTCCATTAAATGTAGCTGTATCTGTGGGAT |
| 5 | 5′-3′ | CGATAAAAAGTCATTGTATCGAGTGATACCGTAACCTACCGTTCCTAGACTATTATAACA |
| 6 | 3′-5′ | GCTATTTTTCAGTAACATAGCTCACTATGGCATTGGATGGCAAGCATCTGATAATATTCT |
| 7 | 5′-3′ | TCTCGGTAAATAGAGTTTCGTGCTTATACTAGATGTAGTCTACGAGATAGACGCTAGATT |
| 8 | 3′-5′ | AGAGCCATTTATCTCAAAGCACGAATATGATCTACATCAGATGCTCTATCTGCGATCTAA |
| 9 | 5′-3′ | AAGTAACGTGAGTAGTATGATCATGTTACGCGAGGATCGTTATCGAGTTACAATAACATA |
| 10 | 3′-5′ | TTCATTGCACTCATCATACTAGTACAATGCGCTCCTAGCAATAGCTCAATGTTATTGTAT |
| 11 | 5′-3′ | TCGGGTTTACTTGATATCAAGCGCGGTTAGAATTGAATACGATGAGACGAATTTATTAGA |
| 12 | 3′-5′ | AGCCCAAATGAACTATAGTTCGCGCCAATCTTAACTTATGCTACTCTGCTTAAATAATCT |
| 13 | 5′-3′ | ATACGAATCTTACGTAGTTTAGTGTCGCTTCACTAAAAGGCTCTATATTCGGATAGTGCA |
| 14 | 3′-5′ | TATGCTTAGAATGCATCAAATCACAGCGAAGTGATTTTCCGAGATATAAGCCTATCACGT |
| 15 | 5′-3′ | GGCTATCATAGAAATGTAGTCGAATCGTAGCATACTCGAATTAGATATCTCTATGCTAAG |
| 16 | 3′-5′ | CCGATAGTATCTTTACATCAGCTTAGCATCGTATGAGCTTAATCTATAGAGATACGATTC |
| 17 | 5′-3′ | CAACGTTGTTATACGTCGTTACCTCAAAATGCGCGTAAAAACCTGTGAACTATTATAAAG |
| 18 | 3′-5′ | GTTGCAACAATATGCAGCAATGGAGTTTTACGCGCATTTTTGGACACTTGATAATATTTC |
| 19 | 5′-3′ | TTGAACTTATGTAATCTGGTAGTATCGAGACAATCGTTACAGCGCCATATGTAATGAGAA |
| 20 | 3′-5′ | AACTTGAATACATTAGACCATCATAGCTCTGTTAGCAATGTCGCGGTATACATTACTCTT |
| 21 | 5′-3′ | TCGTGCAGACTTCTACAACATCGAGTTCTGCAACGTAATAACCGTATGAATAAGACTAGT |
| 22 | 3′-5′ | AGCACGTCTGAAGATGTTGTAGCTCAAGACGTTGCATTATTGGCATACTTATTCTGATCA |
| 23 | 5′-3′ | CTGGTCTTAATCGTCTTGTTAACTAATACGGGCATTTACGAGTCGATAGACATATAATCA |
| 24 | 3′-5′ | GACCAGAATTAGCAGAACAATTGATTATGCCCGTAAATGCTCAGCTATCTGTATATTAGT |
| 25 | 5′-3′ | TGACAACTAGTTTGCAATCGTTATAAGTCGTATTAACGCGAAATTAACCTGCTAGGAACT |
| 26 | 3′-5′ | ACTGTTGATCAAACGTTAGCAATATTCAGCATAATTGCGCTTTAATTGGACGATCCTTGA |
| 27 | 5′-3′ | ATTAGAACTACTATAAATCCGGCGAGATTCTATGGCGCATAACATGATAGACAGAACATT |
| 28 | 3′-5′ | TAATCTTGATGATATTTAGGCCGCTCTAAGATACCGCGTATTGTACTATCTGTCTTGTAA |
| 29 | 5′-3′ | GTTACCGTTTGAATAATAACGGACGGATAACCCTTTGATACATCCCAACGTATAATAAGG |
| 30 | 3′-5′ | CAATGGCAAACTTATTATTGCCTGCCTATTGGGAAACTATGTAGGGTTGCATATTATTCC |
| 31 | 5′-3′ | GTAGAGTATATTGCTTTAATACGACCCCGATAAGCACGATCGTATTAGACATAGATGATA |
| 32 | 3′-5′ | CATCTCATATAACGAAATTATGCTGGGGCTATTCGTGCTAGGATAATCTGTATCTACTAT |
| 33 | 5′-3′ | ATAATTCGTTGACTATAGCACATTTCGATCCTCGTTATGATACCAATGAACGGAAGTCTT |
| 34 | 3′-5′ | TATTAAGCAACTGATATCGTGTAAAGCTAGGAGCAATACTATGGTTACTTGCCTTCAGAA |
| 35 | 5′-3′ | CAGATCGATCGGTTTATATGCGATTTAACGCCGCTTTCATCCTAAAGCGCAAATTTTACA |
| 36 | 3′-5′ | GTCTAGCTAGCCAAATATACGCTAAATTGCGGCGAAAGTAGGATTTCGCGTTTAAAATGT |
| 37 | 5′-3′ | TACGTCAATTCGTGATATGCCTTTCGATTATCATACCGAAGAGTCCTTTAGTAAGTTTAG |
| 38 | 3′-5′ | ATGCAGTTAAGCACTATACGGAAAGCTAATAGTATGGCTTCTCAGGAAATCATTCAAATC |
| 39 | 5′-3′ | GAAACTAGTGAAACAGAGTTCGCTAAGCGTCTAAACTCGAGTTTTTACGAACTAATACAA |
| 40 | 3′-5′ | CTTTGATCACTTTGTCTCAAGCGATTCGCAGATTTGAGCTCAAAAATGCTTGATTATGTT |
| 41 | 5′-3′ | GGTATTGTTCTTATATTCATCGTGACCAGTAACCAATTGATATCGGATTTCGGTTTACAG |
| 42 | 3′-5′ | CCATAACAAGAATATAAGTAGCACTGGTCATTGGTTAACTATAGCCTAAAGCCAAATGTC |
| 43 | 5′-3′ | CTATTTCTCGAAACCGTTAAATCGAAATGTTATGTCCGCTAATCGAACCACTAATCGTTT |
| 44 | 3′-5′ | GATAAAGAGCTTTGGCAATTTAGCTTTACAATACAGGCGATTAGCTTGGTGATTAGCAAA |

In Table 1, a “plus” strand is listed above its reverse-complement strand (“minus” strand). A control nucleic acid molecule as described herein can comprise a duplex of such plus and minus strands. A negative control probe, as described herein, can comprise a sequence that is complementary to either of these strands. As a non-limiting example, a control nucleic acid molecule can comprise a nucleic acid having the sequence identified by SEQ ID NO:1, and the corresponding negative control probe would be identified by SEQ ID NO:2.

In some embodiments, a control nucleic acid molecule can comprise at least one methyltransferase recognition site (i.e., methyltransferase recognition sequence). Non-limiting examples of such methyltransferase recognition sites include CCGG which is recognized by Hpall methylase (New England Biolabs); GGCC which is recognized by Haelll methylase; CpG which is recognized by Sssl; and TCGA which is recognized by Taql methylase (see, e.g., www.neb.com). Other methyltranferase recognition sites, include, for example, CpG, CpA, CpT and CpNpG (see, e.g., Ramsahoye et al. (2000) Proc. Nat. Acad. Sci. 97:5237-5242).

In some embodiments, insert 14 comprises at least 1, 2, 3, 4, 5, 6, 7, 8, 9, 10 or more CpG dinucleotides. In some embodiments, the sequence of insert 14 comprises at least 10%, 20%, 30%, 40%, 50%, 60%, 70%, or 80% CpG dinulceotides. In some embodiments, the sequence of insert 14 comprises about 10% to about 80% CpG dinulceotides.

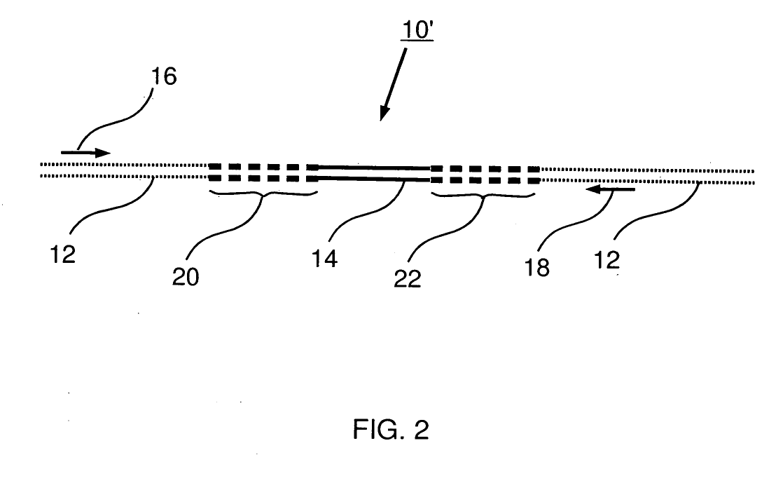

A control nucleic acid construct 10′ can comprise optional insert 20 which can be continguous with insert 14 (FIG. 2). Insert 20 can range in length from 10 to 1000 nt and can comprise a methylation site, such as, for example, a CpG dinucleotide. In some embodiments, the sequence of insert 20 comprises at least 10%, 20%, 30%, 40%, 50%, 60%, 70%, or 80% CpG dinulceotides In some embodiments, insert 20 comprises about 10% to about 80% CpG dinulceotides. In some embodiments, insert 20 comprises about 0 to 100 CpG dinucleotides. Insert 20 can comprise at least one methyltransferase recognition site. In some embodiments, the sequence of insert 20 comprises at least 10%, 20%, 30%, 40%, 50%, 60%, 70%, or 80% of methyltransferase recognition sites.

A control nucleic acid construct 10′ can comprise optional insert 22 which can be continguous with insert 14 (FIG. 2). Insert 22 can range in length from 10 to 1000 nt and can comprise a methylation site, such as, for example, a CpG dinucleotide. In some embodiments, the sequence of insert 22 comprises at least 10%, 20%, 30%, 40%, 50%, 60%, 70%, or 80% CpG dinulceotides In some embodiments, insert 22 comprises about 10% to about 80% CpG dinulceotides. In some embodiments, insert 22 comprises about 0 to 100 CpG dinucleotides. Insert 22 can comprise at least one methyltransferase recognition site. In some embodiments, the sequence of insert 22 comprises at least 10%, 20%, 30%, 40%, 50%, 60%, 70%, or 80% of methyltransferase recognition sites.

In some embodiments, an insert such as insert 20 or insert 22 can have, for example, between about 50 and about 1000 nucleotides, with a combined frequency of appearance of both cytosine and guanine greater than about 60% or about 65%. In some embodiments, an insert can have a length of 300 base pairs and contain 1, 10 or 100 methylation sites, such as, for example, CpG dinucleotides.

In some embodiments, the sequence of at least one of insert 14, insert 20 and insert 22 comprises at least 10%, 20%, 30%, 40%, 50%, 60%, 70%, or 80% of methyltransferase recognition sites. In some embodiments, the sequence of at least one of insert 14, insert 20 and insert 22 comprises about 10% to about 80% methyltransferase recognition sites.

In some embodiments, control nucleic acid constructs as described herein can comprise one or more methyltransferase recognition sites, non-limiting examples of which include CpG, CpA, CpT, CpNpG (where N is any nucleotide), ApG, GpG, and combinations thereof.

In some embodiments, the sequence of at least one of insert 14, insert 20 and insert 22 in a control nucleic acid construct 10′ comprise at least 10%, 20%, 30%, 40%, 50%, 60%, 70%, or 80% CpG dinulceotides. In some embodiments, the sequence of at least one of insert 14, insert 20 and insert 22 in a control nucleic acid construct 10′ comprise at least 10%, 20%, 30%, 40%, 50%, 60%, 70%, or 80% CpA dinulceotides. In some embodiments, the sequence of at least one of insert 14, insert 20 and insert 22 in a control nucleic acid construct 10′ comprise at least 10%, 20%, 30%, 40%, 50%, 60%, 70%, or 80% CpT dinulceotides. In some embodiments, the sequence of at least one of insert 14, insert 20 and insert 22 in a control nucleic acid construct 10′ comprise at least 10%, 20%, 30%, 40%, 50%, 60%, 70%, or 80% ApG dinulceotides. In some embodiments, the sequence of at least one of insert 14, insert 20 and insert 22 in a control nucleic acid construct 10′ comprise at least 10%, 20%, 30%, 40%, 50%, 60%, 70%, or 80% GpG dinulceotides.

In some embodiments, all of the cytosines in the CpGs in a control nucleic acid construct (or amplicon thereof have been methylated. In some embodiments, some of the cytosines in the CpGs in a control nucleic acid construct (or amplicon thereof) have been methylated. In some embodiments, none of the cytosines in the CpGs in a control nucleic acid construct (or amplicon thereof have been methylated.

In some embodiments, all of the methylation sites in insert 14, insert 20 and insert 22 have been methylated. In some embodiments, the metylation sites in insert 14, insert 20 and insert 22 have been partially methylated. In some embodiments, none of the methylation sites in insert 14, insert 20 and insert 22 have been methylated. A methylation site can be non-methylated, hemimethylated or fully methylated.

In some embodiments, the sequences of at least one of insert 14, insert 20 and insert 22 are designed such that they do not hybridize to nucleic acids expected to be in a sample under investigation under stringent conditions. In some embodiments, each of the sequences of insert 14, insert 20 and insert 22 are designed such that they do not hybridize to nucleic acids expected to be in a sample under investigation under stringent conditions. In some embodiments, insert 14 is designed to hybridize to a negative control probe in an array under stringent conditions, and neither insert 20 nor insert 22 hybridize to the array under those same conditions.

In some embodiments, mixtures of different control nucleic acid constructs are provided. In some embodiments, the same vector is used, but with nucleic acid molecules having differing sequences inserted into each of the different constructs in the mixture. In some embodiments, there are provided mixtures of control nucleic acid constructs, wherein at least some of the control nucleic acid constructs in the mixture have different numbers of CpG dinucleotides (or other methyltransferase recognition sites). In some embodiments, there are provided mixtures of same length amplicons obtained from control nucleic acid constructs as described herein, wherein at least some of the amplicons in the mixture have different numbers of CpG dinucleotides (or other methyltransferase recognition sites). In these mixtures, the various different control nucleic acid constructs (or amplicons thereof can all be at the same concentration. In some embodiments, in such a mixture, at least some of the different control nucleic acid construct (or amplicons thereof are at different concentrations.

In some embodiments, the length of a control nucleic acid construct 10 can be in the range of 2 to 10 kilobases, 10 to 20 kilobases, 10 to 50 kilobases, or 10 to 100 kilobases, for example. In some embodiments, the length of al control nucleic acid construct can be greater than 2 kilobases, greater than 10 kilobases, greater than 50 kilobases, greater than 100 kilobases, or longer.

In some embodiments, a control nucleic acid construct as described herein does not include at least one of the following: a homopolymeric run, a poly-A sequence, a T3 promoter site, a T7 promoter, a Tag sequence, a concatenated sequence, concatenated Tag sequences, and an RNA promoter (see, e.g., U.S. Patent Application Publication 20040175719).

A control nucleic acid molecule can be prepared synthetically using any suitable method, such as, for example, the known phosphotriester and phosphite triester methods, or automated embodiments thereof. In one such automated embodiment, dialkyl phosphoramidites are used as starting materials and can be synthesized as described by Beaucage et al. (1981) Tetrahedron Letters 22:1859. A non-limiting exemplary method for synthesizing oligonucleotides on a modified solid support is described in U.S. Pat. No. 4,458,066. Chemical synthesis of DNA can be accomplished using a commercial DNA synthesizer such as for example a DNA synthesizer using the thiophosphate method (Shimazu) or a DNA synthesizer using the phosphoamidite method (Perkin Elmer). In some embodiments, methylated phosphoramidites (e.g., a 5-methylcytosine analog) (see, e.g., Glen Research Corp.) can be used during synthesis. In some embodiments, a control nucleic acid molecule can be chemically synthesized as a single-stranded molecule, and can include a flanking sequence, such as a sequence corresponding to insert 20 and/or insert 22 described hereinabove. In some embodiments, such a single-stranded control nucleic acid molecule can be used as a spiking reagent in methods described herein. It will be apparent that a singled stranded spiking reagent can be synthesized to comprise any desired number or combination of methylated nucleosides, and is not constrained to those sequences required for methylation by methyltransferase.

A control nucleic acid construct can be prepared by incorporating a double-stranded control nucleic acid molecule into an appropriate cloning vector. E. coli or other host cells are transformed using the recombinant vector, and positive transformants are selected using tetracycline resistance or ampicillin resistance as the marker. The cloning vector for preparing a control nucleic acid construct may be any vector capable of independent replication in host cells, and for example a phage vector, plasmid vector or the like can be used. Escherichia coli cells or the like for example can be used as the host cells.

Transformation of E. coli or other host cells can be accomplished for example by a method of adding the recombinant vector to competent cells prepared in the presence of calcium chloride, magnesium chloride or rubidium chloride. When a plasmid is used as the vector, it is desirable to include therein a tetracycline, ampicillin or other drug-resistance gene.

In some embodiments, to prepare a recombinant vector, a nucleic acid fragment (e.g., DNA fragment) of a suitable length is prepared which comprises the control nucleic acid molecule. A recombinant vector is prepared by inserting this control nucleic acid molecule downstream from the promoter of an appropriate expression vector, and this recombinant vector is introduced into appropriate host cells. The aforementioned control nucleic acid molecule is incorporated into the vector so that it may be cloned. In addition to the promoter the vector may contain enhancers and other cis-elements, splicing signals, poly A addition signals, selection markers (such as the dihydrofolic acid reductase gene, ampicillin resistance gene or neomycin resistance gene), ribosome binding sequences (SD sequences) and the like.

Any suitable expression vector can be used in making a control nucleic acid construct as described herein as long as the vector does not have a sequence that interferes with processing or analysis steps as described herein. A vector is generally considered to be an agent that can carry a DNA fragment into a host cell. A wide variety of vectors are available. There are no particular limits on the expression vector as long as it is capable of independent replication in the host cells, and for example plasmid vectors, phage vectors, virus vectors and the like can be used. Non-limiting examples of vectors include double-stranded, linear, or circular molecules. The vector can be a viral nucleic acid. Non-limiting embodiments of suitable vectors include EIA adenovirus, filamentous phage, phage, cosmid, YAC, and lambda phage. Other examples include lambda gt11 (Stratagene; and see, e.g., Young et al. (1983) Proc. Nat. Acad. Sci. USA 80:1194-1198), lambda ZAP, lambda ZAP, lambda DASH, lambda gt101, pDrive Cloning Vector (Qiagen), N15, pQE-30 UA vector, Flexi, pCAT-3, pGEM, PGL2, PG5luc, PGL3, PSP, M13, and PBR322. Non-limiting examples of plasmid vectors include E. coli-derived plasmids (such as pRSET, pBR322, pBR325, pUC118, pUC119, pUC18 and pUC19), B. subtilis-derived plasmids (such as pUB110 and pTP5) and yeast-derived plasmids (such as YEp13, YEp24 and YCp50), examples of phage vectors include gamma-phages (such as Charon4A, Charon21A, EMBL3, EMBL4, gamma-gt10, gamma-gt11 and gamma-ZAP), and examples of virus vectors include animal viruses including retroviruses, vaccinia virus and the like and insect viruses such as baculoviruses and the like.

Any of prokaryotic cells, yeasts, animal cells, insect cells, plant cells or the like can be used as the host cells as long as they can express the nucleic acid contstruct. Individual animals, plants, silkworms or the like can also be used.

When using bacterial cells as host cells, for example Escherichia coli or other Escherichia, Bacillus subtilis or other Bacillus, Pseudomonas putida or other Pseudomonas or Rhizobium meliloti or other Rhizobium bacteria can be used as the host cells. Specifically, E. coli such as Escherichia coli XL1-Blue, Escherichia coli XL2-blue, Escherichia coli DH1, Escherichia coli K12, Escherichia coli JM109, Escherichia coli HB101 or the like or Bacillus subtilis such as Bacillus subtilis M114, Bacillus subtilis 207-21 or the like can be used. There are no particular limits on the promoter in this case as long as it is capable of expression in E. coli or other bacteria, and for example a trp promoter, lac promoter, PL promoter, PR promoter or other E. coli- or phage-derived promoter can be used. An artificially designed and modified promoter such as a tac promoter, lac T7 promoter or let I promoter can also be used.

There are no particular limits on the method of introducing the recombinant vector into the bacteria as long as it is a method capable of introducing DNA into bacteria, and for example electroporation or a method using calcium ions or the like can be used.

When using yeasts as host cells, for example Saccharomyces cerevisiae, Schizosaccharomyces pombe, Pichia pastoris or the like can be used as the host cells. There are no particular limits on the promoter in this case as long as it can be expressed in yeasts, and for example a gall promoter, gal10 promoter, heat shock protein promoter, MFα1 promoter, PHO5 promoter, PGK promoter, GAP promoter, ADH promoter, AOX1 promoter or the like can be used.

There are no particular limits on the method of introducing the recombinant vector into the yeast as long as it is a method capable of introducing DNA into yeast, and for example, the electroporation method, spheroplast method, lithium acetate method or the like can be used.

When using animal cells as host cells, for example monkey COS-7 cells, Vero cells, chinese hamster ovary cells (CHO cells), mouse L cells, rat GH3, human FL cells or the like can be used as the host cells. There are no particular limits on the promoter in the case as long as it can be expressed in animal cells, and for example an SRα promoter, SV40 promoter, LTR (long terminal repeat) promoter, CMV promoter, human cytomegalovirus initial gene promoter or the like can be used.

There are no particular limits on the method of introducing the recombinant vector into the animal cells as long as it is a method capable of introducing DNA into animal cells, and for example the electroporation method, calcium phosphate method, lipofection method or the like can be used.

When using insect cells as host cells, for example Spodoptera frugiperda ovary cells, Trichoplusia in ovary cells, cultured cells derived from silkworm ovaries or the like can be used as the host cells. Examples of Spodoptera frugiperda ovary cells include Sf9, Sf21 and the like, examples of Trichoplusia ni ovary cells include High 5, BTI-TN-5B1-4 (Invitrogen) and the like, and examples of cultured cells derived from silkworm ovaries include Bombyx mori N4 and the like.

There are no particular limits on the method of introducing the recombinant vector into the insect cells as long as it is a method capable of introducing DNA into insect cells, and for example the calcium phosphate method, lipofection method, electroporation method or the like can be used.

A transformant into which has been introduced a recombinant vector having incorporated control nucleic acid construct is cultured by conventional culture methods. Culture of the transformant can be accomplished according to normal methods used in culturing host cells.

For the medium for culturing a transformant obtained as E. coli, yeast or other microbial host cells, either a natural or synthetic medium can be used as long as it contains carbon sources, nitrogen sources, inorganic salts and the like which are convertible by the microorganism and is a medium suitable for efficient culture of the transformant.

Glucose, fructose, sucrose, starch and other carbohydrates, acetic acid, propionic acid and other organic acids, and ethanol, propanol and other alcohols can be used as carbon sources. Ammonia, ammonium chloride, ammonium sulfate, ammonium acetate, ammonium phosphate and other ammonium salts of inorganic or organic acids and peptone, meat extract, yeast extract, corn steep liquor, casein hydrolysate and the like can be used as nitrogen sources. Monopotassium phosphate, dipotassium phosphate, magnesium phosphate, magnesium sulfate, sodium chloride, ferrous sulfate, manganese sulfate, copper sulfate, calcium carbonate and the like can be used as inorganic salts.

Culture of a transformant obtained as E. coli, yeast or other microbial host cells can be accomplished under aerobic conditions such as a shaking culture, aerated agitation culture or the like. The culture temperature is normally 25 to 37° C., the culture time is normally 12 to 48 hours, and the pH is maintained at 6 to 8 during the culture period. pH can be adjusted using inorganic acids, organic acids, alkaline solution, urea, calcium carbonate, ammonia or the like. Moreover, antibiotics such as ampicillin, tetracycline and the like can be added to the medium as necessary for purposes of culture.

When culturing a microorganism transformed with an expression vector using an inducible promoter as the promoter, an inducer can be added to the medium as necessary. For example, isopropyl-β-D-thiogalactopyranoside or the like can be added to the medium when culturing a microorganism transformed with an expression vector using a lac promoter, and indoleacrylic acid when culturing a microorganism transformed with an expression vector using a trp promoter.

Commonly used RPMI1640 medium, Eagle's MEM medium, DMEM medium, Ham F12 medium, Ham F12K medium or a medium comprising one of these media with fetal calf serum or the like added can be used as the medium for culturing a transformant obtained with animal cells as the host cells. The transformant is normally cultured for 3 to 10 days at 37° C. in the presence of 5% CO₂. Moreover, an antibiotic such as kanamycin, penicillin, streptomycin or the like can be added as necessary to the medium for purposes of culture.

Transformants which can use commonly used TNM-FH medium (Pharmingen), Sf-900 II SFM medium (Gibco-BRL), ExCell400, ExCell405 (JRH Biosciences) or the like as the medium for culturing a transformant obtained with insect cells as the host cells are normally cultured for 3 to 10 days at 27° C. An antibiotic such as gentamicin or the like can be added to the medium as necessary for purposes of culture.

A control nucleic acid construct as described herein can be cloned and purified using conventional methods. Any suitable means can be used to insert a control nucleic acid construct into a vector. In some embodiments, a control nucleic acid strand and its reverse-complement strand are synthesized to include additional terminal bases which can be used, after the strands are annealed, to create an overhang which will facilitate ligation into a vector restriction site. For example, a sequence that will re-create a restriction endonuclease site can be incorporated into terminal sequences of control nucleic acid strands facilitating insertion into a vector that has been cleaved with the restriction endonuclease (such as, e.g., EcoR1). Preparation of DNA from bacteria can be accomplished using standard methods (see, e.g., Ausubel, et al.). Lipid and protein can be removed by digestion with proteinase K. Cell wall debris, polysaccharides, and remaining proteins can be removed by selective precipitation with cetyltrimethylammonium bromide (CTAB), and high molecular weight DNA can be recovered from the resulting supernatant by isopropanol precipitation. A cesium chloride gradient may also be utilized. Agarose gel electrophoresis can also be used in the purification.

In some embodiments, the complete sequence of a control nucleic acid construct is used in the methods described herein. In some embodiments a region (section) of a control nucleic acid construct is amplified to produce an amplicon (amplification product) comprising a control nucleic acid molecule, and the amplicon can be used in the methods described herein. In some embodiments, the length of the amplicon can be in the range of about 0.5 kb (kilobases) to about 10 kb, about 1 to about 5 kb, or about 0.5 to about 2 kb. Any suitable amplification method can be used. In some embodiments, the length of a spiking reagent is in the range of about 50% to 200% of the length of the nucleic acids in a sample being analyzed. In some embodiments, the length of a spiking reagent is in the range of about 10% to about 50%, about 10% to about 200%, about 50% to about 150%, or about 80% to about 120% of the length of the nucleic acids in a sample being analyzed. In some embodiments, the length of a spiking reagent is in the range of about 10% to about 200% of the length of the nucleic acids in a sample being analyzed. In some embodiments, the length of a spiking reagent is about 100% of the length of the nucleic acids in a sample being analyzed.

An exemplary amplification method is polymerase chain reaction (PCR). PCR is well known in the biotechnology art and is described in detail in U.S. Pat. No. 4,683,202; Eckert et al., The Fidelity of DNA polymerases Used In The Polymerase Chain Reactions, McPherson, Quirke, and Taylor (eds.), “PCR: A Practical Approach”, IRL Press, Oxford, Vol. 1, pp. 225-244; Andre, et. al. (1977) GENOME RESEARCH, Cold Spring Harbor Laboratory Press, pp. 843-852. In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

In some embodiments, there are provided herein PCR primers capable of amplifying a region of a control nucleic acid construct wherein the region comprises a control nucleic acid molecule. A pair of such primers is shown schematically at 16 and 18 in FIG. 1. Non-limiting examples of forward and reverse PCR primers capable of amplifying a sequence inserted into the EcoR1 site of Lambda gt11 include the following:

| | |
|---|---|
| CTGGATGTCGCTCCACAAA | SEQ ID NO: 45 |
| TTGATCGCCAGATAGTGGTGCTTC | SEQ ID NO: 46 |

“Primer” refers to an oligonuleotide, whether occurring naturally as in a purified restriction digest or produced synthetically, which is capable of acting as a point of initiation of synthesis when placed under conditions in which synthesis of a primer extension product that is complementary to a target nucleic acid strand is induced, i.e., in the presence of nucleotides and an agent for polymerization (such as a DNA polymerase) and at a suitable temperature and pH. The primer is preferably single stranded for maximum efficiency in amplification. Preferably, the primer is an oligodeoxyribonucleotide. The primer must be sufficiently long to prime the synthesis of extension products (referred to herein as “PCR products” and “PCR amplicons”) in the presence of the polymerization agent. Primers are preferably selected to be “substantially” complementary to a portion of the target nucleic acid sequence to be amplified. This typically means that the primer must be sufficiently complementary to hybridize with its respective portion of the target sequence. For example, a primer may include a non-complementary nucleotide portion at the 5′ end of the primer, with the remainder of the primer being complementary to a portion of the target sequence. Alternatively, non-complementary bases or longer sequences can be interspersed into the primer, provided that the primer sequence has sufficient complementarity with a portion of the target sequence to hybridize therewith, and thereby form a template for synthesis of the extension product.

An “amplicon” is a polynucleotide product generated in an amplification reaction.

In some embodiments, the sequence of a vector can be modified using conventional methods of molecular biology to remove C and/or G in a CpG dinucleotide in said vector. Essentially any suitable modification can be made as long as the vector remains functional in the methods herein. For example, the modification can comprise substitution of another base for C and/or another base for G in a CpG dinucleotide. For example, an A, T, or G can be substituted for the C and/or an A, T, or C can be substituted for the G. In some embodiments, for an amplicon comprising a region comprising a nucleic acid control sequence within a control nucleic acid construct, the sequence of the vector can be modified, using conventional methods, and/or the PCR primers can be designed, such that no CpG dinucleotides originating from the vector sequence occur in the resulting amplicon; only CpG dinucleotides originating from an insert, such as insert 14, inset 20 or insert 22, are present in the resulting amplicon. In some embodiments, for an amplicon comprising a region comprising a nucleic acid control molecule within a control nucleic acid construct, the sequence of the vector can be modified, using conventional methods, and/or the PCR primers can be designed, such that the number of methltransferase recognition sites (e.g., CpG dinucleotides) originating from the vector sequence that occur in the resulting amplicon is reduced compared to the un-modified vector sequence.

Compositions of control nucleic acid constructs, or amplicons thereof, can be prepared having varied degrees of saturation of methylation and can be used as spiking reagents as described herein. For example, an amplicon of a control nucleic acid construct can be prepared comprising a plurality of CpG sites within an inserted sequence as described above. The amplicon can be split into two batches: in one batch, all of the plurality of CpG sites (and/or methyltransferase recognition sites) are methylated (using, e.g., in vitro techniques), and in the other batch, none of the CpG sites (and/or methyltransferase recognition sites) have been methylated. These two batches can be mixed in essentially any proportion. For example, compositions having any selected degree of saturation of methylation, from 0 to 100%, can be prepared. In some embodiments, partial methylation of a control nucleic acid construct (or amplicon thereof) can be achieved by limiting an in vitro methylation reaction such that only a percentage of the control nucleic acid construct (or amplicon) is methylated.

In vitro methylation can be effected using conventional methods (see, e.g., U.S. Pat. Nos. 6,605,432; 6,960,434; U.S. Patent Application Publications 20050196792 and 20050233340; WO2005123942; Kimura et al. (2005) Nuc. Acids Res. 33:e46; Schumacher et al. (2006) Nuc. Acids Res. 34:528-542).

### Utility

The spiking reagents as described herein can be used with any conventional method for determining the methylation status of CpG dinucleotides. In some embodiments, such reagents can be used as normalization controls in DNA methylation analysis experiments. These reagents can be used to assess system specificity, sensitivity, and dynamic range, and can be used in assay development, in product development and validation, and for quality control.

In some embodiments, a control nucleic acid construct as described herein can be used as a spiking reagent in methods that employ one or more sample preparation and analysis steps. For example, a control nucleic acid construct as described herein can be mixed with a nucleic acid sample, and the mixture subjected to one or more steps such as fragmentation, immunoprecipitation, amplification, labeling, and array hybridization. Any conventional method of fragmentation can be used, including mechanical shearing or enzymatic cleavage.

In some embodiments, methylated spiking reagents can be spiked into a genomic DNA sample that is being assayed for methylation (e.g., CpG methylation) and the sample can be subjected to a shearing or fragmentation process. In some embodiments of such an assay, the sheared methylated DNA can be isolated using an antibody (e.g., an antibody to 5-methyl-cytosine) as further described herein. The analysis for the isolation of the control can utilize PCR detection methods, or the isolated DNA can be fluorescently labeled for microarray hybridization. Methylated spike-in reagents can thus provide a means for ensuring that the immunoprecipitation was effective. The amount of a methylated DNA species isolated using the antibody in an immunoprecipitation (IP) can be compared to the amount of that same DNA species that is present in the material that was input into the immunoprecipitation. This provides an estimate of the efficiency of the IP and the sensitivity of the assay. The degree of recovery of the methylated spike-in reagents from a particular assay can then serve as a means for assay qualification and/or calibration of the efficiency of isolation.

The presently disclosed spike-in compositions can be used in conjunction with a variety of conventional methods for determining the methylation status of CpG dinucleotides. One such method involves bisulfite nucleotide sequencing. This method, developed by Frommer and colleagues (Proc. Natl. Acad. Sci. (1992) 89:1827-1831), relies on the ability of sodium bisulfite to deaminate non-methylated cytosine residues into uracil in genomic DNA. In contrast, methylated cytosine residues are resistant to this modification. After bisulfite treatment, target DNA is cloned and sequenced and the methylation status of individual CpG sites is then analyzed by comparing the obtained sequence with the sequence of the same DNA that has not been treated with bisulfite. Using this conventional bisulphite modification method, many investigators have addressed the importance of promoter CpG hypermethylation in the regulation of specific gene transcription in cancer (e.g., Hiltunen et al. (1997) Int. J. Cancer 70:644-648; Stirzaker et al. (1997) Cancer Res. 57:2229-2237; Melki et al. (1998) Leukemia 12:311-316).

Another bisulphate modification assay for the methylation status of CpGs relies on sets of PCR primers that, although designed for the same target DNA, are specific to either the converted (i.e. unmethylated Cs changed to Ts) or unconverted (i.e. methylated Cs remain Cs) nucleotides in a bisulfite treated sample (Herman et al. (1996) Proc. Natl. Acad. Sci. USA. 93:9821-9826). The presence of methylation in a region of interest is detected by the presence of PCR products with the set of primers that are specific for unconverted sequences.

In some embodiments, the methylated spiking reagents can also be applied to methods utilizing methylation sensitive restriction enzymes as part of the assay. Restriction endonucleases (such as, e.g., Hpall and BstUI) do not cut DNA that has been methylated at cytosines that are within the enzyme recognition sequence. Spiking reagents with one or several of these restriction enzyme sequences can be generated as described herein and then methylated. The degree of digestion of the spiking reagents can be monitored by PCR or by oligonuclotide probes which span the cut site. In some embodiments, a methylation-sensitive restriction endonuclease and a methylation-insensitive isoschizomer of that endonuclease are used to differentiate between methylated and unmethylated cytosines in the recognition motif for the endonucleases. In some embodiments, the methylation status of a particular CpG island can be assessed by determining whether the CpG island is cleaved by a methylation sensitive enzyme that recognizes a methylated cytosine-containing motif within the CpG island. Separate aliquots of the same genomic DNA can be digested with each of the enzymes, and the methylation status of a CpG island in the DNA can be deduced by detecting the presence or absence of specific DNA restriction fragments. In some methods, Southern blotting is used, which involves separating the digested DNA fragments on the basis of size (e.g., by gel electrophoresis), and hybridization with a labeled probe that detects the DNA fragments of interest. In other methods, a post-digest PCR amplification step is performed where a set oligonucleotide primers, one on each side of the methylation sensitive restriction site, is used to amplify the digested DNA. If the methylation sensitive enzyme does not digest a CpG island because the CpG island is methylated, PCR amplification products will be detected.

Further techniques, such as differential methylation hybridization (DMH) (Huang et al. (1999) Human Mol. Genet. 8:459-70); Not 1-based differential methylation hybridization (see e.g., WO 02/086163 A1); restriction landmark genomic scanning (RLGS) (Plass et al. (1999) Genomics 58:254-62); methylation sensitive arbitrarily primed PCR (AP-PCR) (Gonzalgo et al. (1997) Cancer Res. 57:594-599); and methylated CpG island amplification (MCA) (Toyota et. al. (1999) Cancer Res. 59: 2307-2312), can also be used. Other examples of a method of assessing CpG methylation include those disclosed in U.S. Patent Application Publication 20050233340 and in U.S. patent application Ser. No. 11/390,828, filed Mar. 28, 2006.

Another technique used in analysis of CpG methylation comprises methylated DNA immunoprecipiation (MeDIP) (see, e.g., Weber et al. (2005) Nature Genetics 37:853-862; Keshet et al. (2006) Nature Genetics 38:149-153; WO2005123942). In some embodiments, there are provided herein methods for enriching methylated nucleic acid fragments in a sample of nucleic acid fragments comprising the steps of: spiking the nucleic acid fragments with a control nucleic acid construct (or PCR amplification product thereof) as described herein; contacting the sample of nucleic acid fragments with an antibody specific to a methylated nucleoside under conditions suitable for binding of the antibody to the methylated nucleoside; and selecting nucleic acid fragments bound to the antibody. In some embodiments, prior to selecting the nucleic acid fragments bound to the antibody specific to a methylated nucleoside, the methylated and non-methylated fragments can be separated on the basis of binding of the methylated fragments to the antibody. In some embodiments, the methods may further comprise a step of separating the strands of any double-stranded nucleic acid fragments in the spiked sample to form a sample of single-stranded nucleic acid fragments, before contacting the sample of single-stranded nucleic acid fragments with an antibody specific to a methylated nucleoside.

In some embodiments there are provided a methods for characterizing or identifying methylated nucleic acid fragments from a sample of nucleic acid fragments, the method further including the step of: characterizing one or more of the methylated nucleic acid fragments.

By “enrichment” is meant an increase in the proportion of a particular category of nucleic acid fragment in or from a sample of nucleic acid fragments. The enrichment is at least 1.1, 1.5, 5, 10, 20, 30, 50, or 100 fold, for example.

In some embodiments, there are provided methods of determining the distribution of DNA methylation in disease and thereby targets for therapeutic intervention as well as diagnostics, prognostics and surrogate markers useful in the fight against cancer and other diseases.

Although the above described immunoprecipitation method may be applied to a sample of any type of nucleic acid, in some embodiments, the nucleic acid is DNA. Examples of methylated nucleosides include methylated cytidine (e.g., 5-methyl cytidine), methylated adenosine (e.g., 6-methyl adenosine) and methylated guanosine (7-methyl guanosine). In some embodiments, the methylated nucleoside is methyl cytidine (e.g., 5-methyl cytidine).

The sample may be any which it is desired to be analyzed. The skilled person can readily determine how to fragment nucleic acid to produce a sample of nucleic acid fragments. For example, genomic DNA may be fragmented using shearing (e.g., by sonication) or digestion with restriction enzymes such as Alul. Once obtained, the sample of nucleic acid fragments can be suspended in a liquid (e.g., a buffer suitable for antibody binding).

Denaturation of the strands is most readily done by heating the nucleic acid. The skilled person can readily determine a temperature and length of heating time suitable for denaturing the nucleic acid that they are interested in. Heating to 95° C. for 10 minutes has been found to be effective for DNA for use in the present disclosure.

Antibodies specific to many methylated bases are available commercially. For example a mouse monoclonal antibody against m5C is available from Eurogentec S. A. (Belgium) and a rabbit polyclonal serum is available from Megabase Research Products (USA). Polyclonal rabbit antisera against other methylated bases (6-methyladenosine and 7-methylguanosine) are available (Megabase Research Products, USA). Alternatively antibodies specific to methylated bases can be made using conventional techniques (see, e.g., Roitt et al. in “Immunology 5th edition” (1997) Moseby International Ltd, London).

The term “antibody” as used herein should be construed as covering any specific binding substance having a binding domain with the required specificity. Thus, this term covers antibody fragments, derivatives, functional equivalents and homologues of antibodies, including any polypeptide comprising an immunoglobulin binding domain, whether natural or synthetic. Chimeric molecules comprising an immunoglobulin binding domain, or equivalent, fused to another polypeptide are therefore included. Cloning and expression of chimeric antibodies are described in EP-A-0120694 and EP-A-0125023. For example, it has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. (1989) Nature 341:544-546) which consists of a VH domain; (v) isolated CDR regions; (vi) F (ab′)₂ a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science 242:423-426; Huston et al. (1988) Proc. Natl. Acad. Sci. USA, 85:5879-5883); (viii) bispecific single chain Fv dimers (PCT/US92/09965) and (ix) “diabodies”, multivalent or multispecific fragments constructed by gene fusion (WO94/13804; Holliger et al. (1993) Proc. Natl. Acad. Sci. USA 90:6444-6448). Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804). In some embodiments, the antibody is specific for methylcytidine. In some embodiments, the antibody is specific for 5-methylcytidine. The skilled person can readily determine the conditions suitable for binding of the first antibody to the methylated nucleoside in a liquid phase. In particular, it is important to maintain an appropriate ionic balance in the sample so that the antibody can bind effectively to the methylated nucleoside. For example, the pH of the sample can be controlled by addition of suitable buffers such as sodium phosphate, which will maintain the pH at approximately 7.0. Salts, such as sodium chloride may also be added to the buffer and/or the sample. The sample can be maintained at approximately 1 to 5° C. while contacting it with the nucleic acid.

Binding of methylated nucleic acid to the first antibody ‘tags’ the methylated nucleic acid. This ‘tagging’ allows methylated nucleic acid to be separated from non-methylated nucleic acid.

In some embodiments, prior to the selection step, methylated and nonmethylated nucleic acid fragments are separated on the basis of binding of the first antibody to the methylated nucleoside. This may be done by any method known to those skilled in the art. In some embodiments, the separation is performed by attaching or binding the antibodies to a solid phase or substrate (the terms are used interchangeably) and separating this solid phase from the sample liquid phase. Thus addition of a solid substrate that binds specifically to the first antibody facilitates the separation of methylated nucleic acid from non-methylated nucleic acid. Specific binding of the solid substrate to the first antibody can be achieved by using a solid substrate that comprises a second antibody specific for the first antibody. For example, if the first antibody (i.e. the antibody specific to a methylated nucleoside) is a mouse anti-m5C antibody, a goat anti-mouse antibody would be suitable. A solid substrate in the form of beads can be used. For example, magnetic beads such as Dynabeads (Dynal Biotech) allow simple separation of methylated and non-methylated nucleic acid as the beads (and therefore the nucleic acid bound to them) can be easily removed from a sample using a magnet. Alternatively, the solid substrate could be separated from the non-bound nucleic acid using techniques such as centrifugation and/or filtration. The skilled person can readily determine a suitable way to separate the solid substrate he is using from non-bound (i.e., non-methylated) nucleic acid.

Prior to characterizing the methylated nucleic acid fragments, it is desirable to detach the methylated nucleic acid from the first antibody (and the solid substrate if used). The skilled person can readily determine such detaching methods in which nucleic acid is not damaged during the detaching process. For example, a nucleic acid fragment may be detached from an antibody by digesting the antibody. This may be achieved by incubating the nucleic acid fragments bound to the first antibody with a proteinase such as Proteinase K. Slightly altering the pH around the nucleic acid bound to the first antibody may weaken the binding between the antibody and methylated nucleic acid, further facilitating detachment. This may be achieved by adding a suitable buffer (e.g., 50 mM Tris pH 8.0) to the methylated nucleic acid and antibody bound to it. The skilled person can readily determine other suitable ways to do this. EDTA (Ethylenediaminetetraacetic acid) and SDS (sodium dodecyl sulphate) may also be added to the buffer. Once it has been detached from the first antibody and the solid substrate, the methylated nucleic acid can be analyzed further—for example to determine the amount present, all or part of the sequence of the methylated fragment and/or the sequence or position of the methylation site. This step may be preceded by further treatment of the nucleic acid. For example where the methylated nucleic acid is DNA it may be extracted (e.g., in phenol and chloroform) and subsequently precipitated (e.g., with ethanol).

Conventional nucleic acid analysis techniques can then be applied to the methylated nucleic acid. For example, the presence of sequences of interest in the methylated nucleic acid may be determined using techniques such as PCR, slot blots, microarrays etc. such as are well known to those skilled in the art. For example analysis may employ a microchip system comprising a microarray of oligonucleotides or longer DNA sequences as described herein. Sample nucleic acid (e.g., fluorescently labeled) may be hybridized to the oligonucleotide array and sequence specific hybridization may be detected. As a control, a sample that has not undergone enrichment can be similarly analyzed compared to the enriched sample.

Since the nucleic acid fragments isolated using the methods described above can be analyzed by either standard PCR or slot blot hybridization this method can be applied to large-scale (genome-wide) analysis using microarrays. Thus there are provided methods of characterizing the methylation status of a DNA sample (for example from an organism genome) comprising: (i) fragmenting the genome (ii) performing a method as described above. By “methylation status” is meant whether, and/or to what extent, the nucleic acid sequence is methylated. The extent of methylation may be measured as which nucleotides in the sequence are methylated and/or the proportion of nucleotides in the sequence which are methylated.

In some embodiments the present methods may be used for detecting differentially methylated alleles in a sample—for example of imprinted genes. “Imprinted genes” are genes whose alleles have different expressivity or penetrance depending on whether they are inherited from the male or the female parent. Imprinting can be both developmental-stage specific or tissue specific. If the maternal and paternal alleles of a gene are differentially methylated, they will be enriched to differing extents in a sample of nucleic acid subjected to the methods of the present disclosure. An example of an imprinted gene whose alleles are differentially methylated is the H19 ICR in mice. This locus contains a CpG island. This CpG island is not methylated in the maternal allele, but methylated in the paternal allele. When applied to a sample of fragments of the mouse genomic DNA, the methods of the present disclosure will enrich the paternal allele but not the maternal allele. The skilled person can readily determine a suitable technique for determining whether the maternal or paternal allele that has been enriched in the sample. The use of a ‘marker’ for either the maternal or the paternal allele is particularly useful. For example, the H19 ICR allele from Mus spretus contains a polymorphic SacI restriction site that is not present in the Mus musculus domesticus H19 ICR allele. Thus, a domesticus×spretus hybrid will have one allele with the SacI restriction site and one without. PCR amplification using primers for the H19 ICR followed by treatment of the PCR product with SacI results in a single 200 bp fragment for the domesticus allele and two 100 bp fragments for the spretus allele. The size of the fragments obtained from an ‘enriched’ sample therefore shows whether the maternal, paternal or both alleles have been enriched.

Aberrant DNA methylation may result in increased expression of proto-oncogenes or decreased expression of tumor suppressor genes and is associated with many human carcinomas. The methods of the present disclosure may be used to screen and identify aberrant nucleic acid methylation sites associated with disease states, or for diagnosis or prognosis of disease or disease progression e.g. in cancer. Novel aberrant nucleic acid methylation sites associated with disease states may be identified by performing the methods of present disclosure on nucleic acid samples from diseased and nondiseased individuals and comparing the results. There are provided methods of diagnosis in an individual of a disease associated with methylation of a specific nucleic acid sequence, comprising: performing methods as described above on a nucleic acid sample from the individual to characterize whether the specific nucleic acid sequence is methylated, and, correlating the result with the disease state of the individual. The detection of changes in nucleic acid methylation can be made over time (e.g. to relate this to clinical history, and hence the diagnosis or prognosis of a disease associated with alterations in methylation of a nucleic acid sequence). Such methods may include the steps of: obtaining a sample of nucleic acid fragments from a patient at least two time points; carrying out the disclosed methods on each sample of nucleic acid fragments for each time point to characterize whether, and/or to what extent, the nucleic acid sequence is methylated. The sample of nucleic acid fragments can be obtained from a patient using the following protocol: obtaining a tissue specimen from the patient; extracting nucleic acid from each tissue specimen to provide a sample of nucleic acid; fragmenting the sample of nucleic acid to give a sample of nucleic acid fragments; The method for detection of changes in nucleic acid methylation over time may also further comprise recording the clinical symptoms of a disease observed in the patient at each time point, and comparing the clinical symptoms recorded at each time point with the methylation status of the nucleic acid sequence of interest at each time point. The method for detection of changes in nucleic acid methylation may be carried out in any appropriate order. For example, the extraction and analysis steps may be carried out at or shortly after each time point. Alternatively, the specimens or samples may be stored and extraction of nucleic acid and/or comparison of the recorded clinical symptoms with methylation status carried out for a plurality of samples together. For example tissue specimens may be frozen or fixed in formalin for storage.

DNA immunoprecipitation (MeDIP), as described above, can be combined with large-scale analysis using DNA microarrays. In carrying out a hybridization analysis, an enormous number of array designs are possible. In some embodiments, a high density array will include a number of probes that specifically hybridize to the nucleic acids in a sample under analysis. In addition, the array can include one or more negative control probes as described hereinbelow. A control nucleic acid molecule (e.g., insert 14) which is inserted into a control nucleic acid construct, as described herein, is perfectly complementary to a negative control probe.

In some embodiments, the signal obtained from binding of a labeled control nucleic acid molecule to an array can provide a control for variations in hybridization conditions, label intensity, reading efficiency, linearity of signal response, and other factors that can cause the signal of a perfect hybridization to vary between arrays. Gradient effects or “trends” are those in which there is a pattern of expression signal intensity which corresponds with specific physical locations on the substrate of the array and which may typically be characterized by a smooth change in the expression values from one location on the array to another. The signal obtained from binding of a labeled control nucleic acid molecule can provide a control for monitoring the uniformity of a microarray, and can be used detrending signal intensity data. Since the control nucleic acid construct is present during processing steps, it can aid in the evaluation of the overall process.

As further described below, negative control probes can be localized at any position in an array or at a multiple positions throughout the array to control for spatial variation in hybridization efficiency. In some embodiments, the negative control probes are located at the corners or edges of the array as well as in the middle. In some embodiments, an array can be divided into a plurality of quadrants or areas, and one or more negative control probes can be randomly located within each of the quadrants or areas.

Negative Control Probes

Some embodiments of methods disclosed herein can be used to generate negative control probe sequences. The term “negative control probe sequence” as used herein includes sequences of bases that can be deposited on an array and serve as a negative control during use of the array.

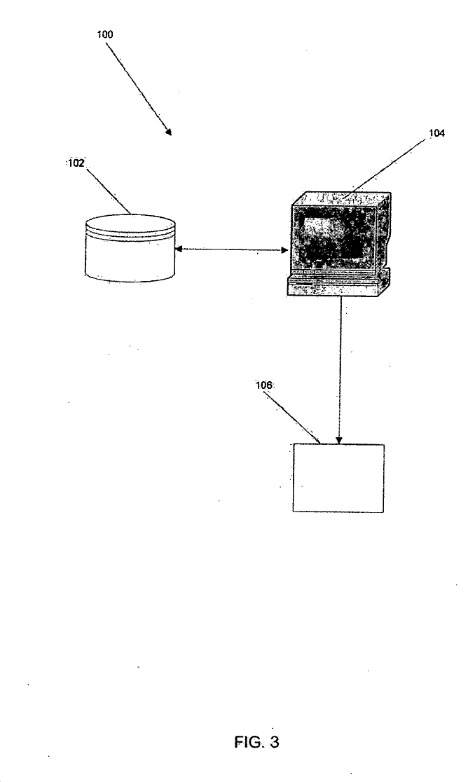

Referring now to FIG. 3, a schematic diagram of an exemplary system 100 for manufacturing arrays is shown. A computing system 104 is in electronic communication with a database 102 and an array printer 106. In some embodiments, the computing system 104 directs the operations of the array printer 106. It will be appreciated that in some embodiments the computing system 104 is part of the array printer 106. However, in some embodiments, the computing system 104 and the array printer 106 are separate. In addition, it will be appreciated that in some embodiments the database 102 is part of the computing system 104. However, in some embodiments, the database 102 and the computing system 104 are separate. The computing system 104 can query the database 102 as desired to retrieve data on probe sequences or on known sequences.

The array printer 106 can perform various steps to generate features of biopolymer probes (e.g., nucleic acids) on the array substrate. Exemplary array manufacturing machines and methods are described in U.S. Pat. Nos. 6,900,048; 6,890,760; 6,884,580; and 6,372,483. In some embodiments, the array printer 106 uses inkjet technology. In some embodiments, the array printer 106 prints spots of pre-synthesized nucleotide sequences onto the array substrate. In some embodiments, the array printer 106 can be used for in situ fabrication, where nucleotide sequences are built on the array one base at a time. Embodiments of the array printer 106 can also include those that use photolithographic methods to deposit nucleotide sequences onto the array substrates. Some embodiments of methods described herein are performed as a part of the array manufacturing process. However, some embodiments of methods described herein are performed separately from the array manufacturing process.

Some embodiments described herein are implemented as logical operations in a computing system, such as the computing system 104. The logical operations can be implemented (1) as a sequence of computer implemented steps or program modules running on a computer system and (2) as interconnected logic or hardware modules running within the computing system. This implementation is a matter of choice dependent on the performance requirements of the specific computing system. Accordingly, the logical operations making up the embodiments described herein are referred to as operations, steps, or modules. It will be recognized by one of ordinary skill in the art that these operations, steps, and modules can be implemented in software, in firmware, in special purpose digital logic, and any combination thereof without deviating from the spirit and scope of the claims attached hereto. This software, firmware, or similar sequence of computer instructions can be encoded and stored upon computer readable storage medium and can also be encoded within a carrier-wave signal for transmission between computing devices.

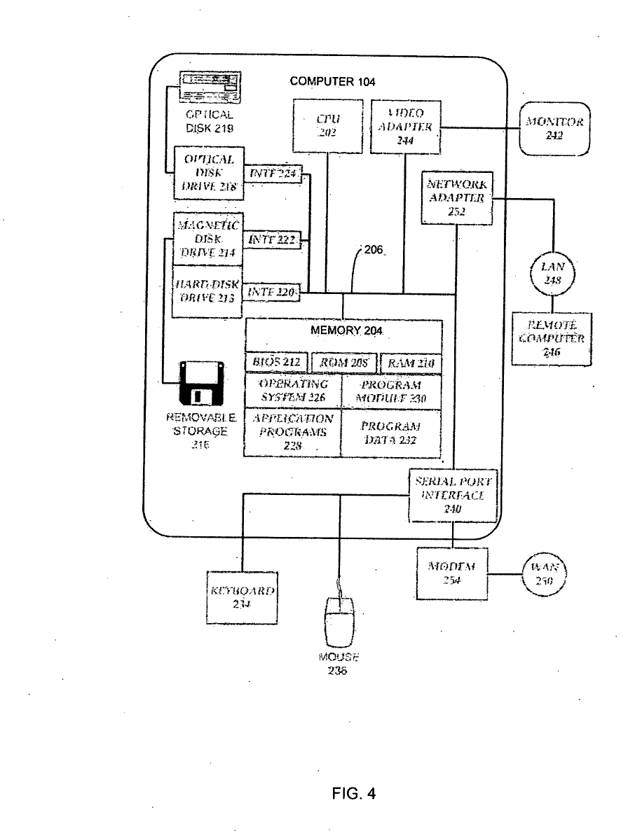

Referring now to FIG. 4, an exemplary computing system 104 is illustrated. The computing system 104 illustrated in FIG. 4 can take a variety of forms such as, for example, a mainframe, a desktop computer, a laptop computer, a hand-held computer, or any other programmable device. In addition, although computing system 104 is illustrated, the systems and methods disclosed herein can be implemented in various alternative computer systems as well.

The computing system 104 includes a processor unit 202, a system memory 204, and a system bus 206 that couples various system components including the system memory 204 to the processor unit 202. The system bus 206 can be any of several types of bus structures including a memory bus, a peripheral bus and a local bus using any of a variety of bus architectures. The system memory includes read only memory (ROM) 208 and random access memory (RAM) 210. A basic input/output system 212 (BIOS), which contains basic routines that help transfer information between elements within the computing system 104, is stored in ROM 208.

The computing system 104 further includes a hard disk drive 213 for reading from and writing to a hard disk, a magnetic disk drive 214 for reading from or writing to a removable magnetic disk 216, and an optical disk drive 218 for reading from or writing to a removable optical disk 219 such as a CD ROM, DVD, or other optical media. The hard disk drive 213, magnetic disk drive 214, and optical disk drive 218 are connected to the system bus 206 by a hard disk drive interface 220, a magnetic disk drive interface 222, and an optical drive interface 224, respectively. The drives and their associated computer-readable media provide nonvolatile storage of computer readable instructions, data structures, programs, and other data for the computing system 104.

Although the example environment described herein can employ a hard disk 213, a removable magnetic disk 216, and a removable optical disk 219, other types of computer-readable media capable of storing data can be used in the example system 104. Examples of these other types of computer-readable mediums that can be used in the example operating environment include magnetic cassettes, flash memory cards, digital video disks, Bernoulli cartridges, random access memories (RAMs), and read only memories (ROMs).

A number of program modules can be stored on the hard disk 213, magnetic disk 216, optical disk 219, ROM 208, or RAM 210, including an operating system 226, one or more application programs 228, other program modules 230, and program data 232.

A user can enter commands and information into the computing system 104 through input devices such as, for example, a keyboard 234, mouse 236, or other pointing device. These and other input devices are often connected to the processing unit 202 through a serial port interface 240 that is coupled to the system bus 206. Nevertheless, these input devices also can be connected by other interfaces, such as a parallel port, game port, or a universal serial bus (USB). An LCD display 242 or other type of display device is also connected to the system bus 206 via an interface, such as a video adapter 244.

The computer system 104 can operate in a networked environment using logical connections to one or more remote computers, such as a remote computer 246. The remote computer 246 can be a computer system, a server, a router, a network PC, a peer device or other common network node, and typically includes many or all of the elements described above relative to the computer system 104. The network connections include a local area network (LAN) 248 and a wide area network (WAN) 250. When used in a LAN networking environment, the computer system 104 is connected to the local network 248 through a network interface or adapter 252. When used in a WAN networking environment, the computing system 104 typically includes a modem 254 or other means for establishing communications over the wide area network 250, such as the Internet. In a networked environment, program modules depicted relative to the computing system 104, or portions thereof, can be stored in the remote memory storage device. It will be appreciated that the network connections shown are examples and other means of establishing a communications link between the computers can be used.

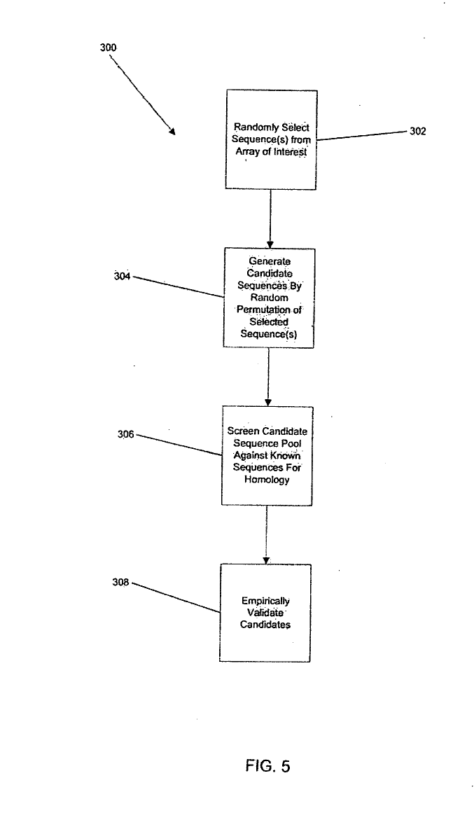

Referring now to FIG. 5, a flowchart 300 is provided illustrating operations that are performed in some embodiments. First, one or more biological probe sequences of interest are randomly selected from an array of interest 302. As used herein, the term “biological probe sequences” includes those sequences of a set of sequences that are designed to hybridize with target molecules (also referred to as biologically occurring molecules), such as nucleotide sequences, that can be present in a sample. Such sequences can be included on a chemical array. Next, a pool of candidate sequences is generated by randomly permuting the bases (or nucleotides) of each selected biological probe sequences 304. The term “permuting” as used herein shall mean to change the order or arrangement of bases within a sequence. One or more screening operations can then performed on the pool of candidate sequences. As an example of one screening operation, the candidate sequences are screened for similarity against known biological sequences of genome or transcriptome of the organism of interest to eliminate those having significant similarity with any known biological sequence 306. The best-alignment of a 60-mer negative control sequence to the human genomic sequence should contain no contiguous hits of more than 20 consecutive bases or about 33% of the probe sequence as determined by a BLAST search using default parameters. Using ProbeSpec with a index-seed size of 10 there should be hits with fewer than 20 mismatches across the length of the probe for the nearest hit in the genome.

The organism of interest is the organism, or any of the organisms, for which the array is designed to analyze samples from. Individual screening operations are performed by themselves or in addition to other screening operations. Then, in some embodiments, the remaining candidate sequences are empirically validated on a test array 308. For example, candidate sequences can be synthesized and then put on a test array (or synthesized in situ) and then the candidate sequences can be tested for hybridization with a test sample. Operations performed in some embodiments will now be discussed in greater detail.

Some embodiments include random selection of biological probe sequences from a set of sequences (e.g., such as a plurality of sequences designed for inclusion on a chemical array of interest). The array of interest is the particular array for which negative control probes are being designed. The selected biological probe sequences then serve as the starting point from which candidate probe sequences are generated (as further described below). In some embodiments, when biological probe sequences are used as the starting point, the resulting candidate probe sequences will match the base composition (e.g., A/T/G/C %) of the biological probe sequences in the array of interest. In some embodiments, the resulting candidate probes can be used to more accurately measure both residual spatially varying background as well as the sequence specific background variations. In some embodiments, by randomly choosing the biological probes to use for generating the candidate probes, the resulting negative control probe sequences have base compositions and thermodynamic properties that closely represent those distributions for the biological probes themselves.

In some embodiments, screening can include screening the candidate sequences for base composition properties such as for A/C/T/G content, the presence or absence of homopolymeric runs, screening for hairpin loops or for thermodynamic characteristics such as for melting temperature. In general, each screening operation reduces the pool of potential candidate sequences. Methods of screening according to such characteristics are described in U.S. patent application Ser. No. 11/232,817, filed Sep. 21, 2005, incorporated by reference herein.

Arrays can include any desired number of biological probe sequences. By way of example, arrays can include 10 s, 100 s, 1,000 s, or 10,000 s of different biological probe sequences. Any desired number of the biological probe sequences can be randomly selected. The desired number can depend on the number of biological probe sequences in the array of interest. In some embodiments, the number of biological probe sequences selected is equal to between about 0.1% and 20% of the biological probe sequences on the array of interest.

It will be appreciated there are many ways of randomly selecting individuals from among a group. By way of example, different biological probe sequences can be assigned different reference numbers and then a subset of the reference numbers can be randomly or pseudo-randomly selected. The term “random” as used herein shall include pseudo-random unless indicated to the contrary. Techniques of random number selection can include lottery methods, the use of random number tables, entropy approaches, and the like. It will also be appreciated that there are many ways of using computer systems to automatically generate random numbers. Further, techniques for generating random numbers can be implemented in many different programming languages. After random selection of biological probe sequences, the selected sequences can then used as the starting point for candidate probe generation.

In some embodiments, nucleotide base sequences are represented by the letters A/T/G/C. It will be appreciated that these letters correspond to the bases occurring in DNA (adenine, thymine, guanine, and cytosine). However, in some embodiments, other letters are used corresponding to components of other biopolymers, such as RNA or polypeptides. In addition, in some embodiments, letters are used corresponding to artificial components such as non-naturally occurring bases or peptides. As used herein the term “bases” or “monomer units” or “letters” can be used interchangeably though in specific contexts as will be apparent, the term “bases” or “monomer units” will refer to the chemical moieties, while “letters” will refer to a representation of the former.

Some embodiments include methods of generating candidate probe sequences. The term “candidate probe sequences” as used herein includes generated sequences that are later subject to one or more screening steps in order to produce negative control probe sequences. Biological probe sequences selected from an array of interest can serve as the starting point for the generation of a pool of candidate probe sequences. By way of example, the selected biological probe sequences can be randomly permuted to form a pool of candidate probe sequences. There are many techniques of random sequence permutation that can be used. By way of example, the letters (corresponding to bases) of a given selected biological probe sequence can be tallied with regard to the total number of each letter present. By way of example, assuming the selected biological probe sequences are 60 bases in length, a given selected biological probe sequence can be found to contain the following composition of bases: 13 A, 16 T, 15 G, and 16 C. A permuted random sequence can then be generated using this group of letters by randomly selecting one letter out of the group for each position in the permuted sequence until all of the 60 letters are used. In this case, the resulting permuted sequence would still contain a total 60 letters (specifically 13 A, 16 T, 15 G, and 16 C) but the sequence of letters would be different than the sequence of letters in the original selected biological probe sequence. It will be appreciated that there are many other techniques that can be used for generating random permuted sequences based on a given starting sequence.

The total number of possible unique random permutations depends on the total length of the sequence and the composition of different letters within the sequence. However, in the example of a sequence that is 60 bases in length having a relatively even distribution of bases, it will be appreciated that a very large number of random permutations are possible. It is estimated that only a fraction of these randomly generated permutation sequences are found within the sequences of all living organisms. An even smaller fraction would be found with the sequences of a given organism, such as the organism of interest. For any given length of random sequence generated, those that are found within the sequences of the organism of interest can be removed from the candidate pool through similarity screening, in silico, as described further below and/or by empirical testing (e.g., in a hybridization experiment).

In some embodiments, the pool of candidate sequences generated is screened for sequence similarity against the entire genome (for methylation analysis) or the entire transcriptome (for expression arrays) of an organism from which samples to be tested will be obtained (organism of interest). The term “sequence similarity” as used herein shall refer to the degree to which two sequences are similar in their base sequence. Sequence similarity can be quantified in various ways known to those of skill in the art. Eliminating candidate sequences from the pool that have substantial similarity to sequences of an organism of interest helps to ensure that candidate sequences will be chosen that will function as negative controls. Similarity screening can be performed using many different tools available to those of skill in the art. A possible example includes determining similarity using the BLASTN program available at the website for the National Center for Biotechnology Information (NCBI). The BLASTN program uses the heuristic search algorithm BLAST (Basic Local Alignment Search Tool) to compare a nucleotide sequence (N) against a nucleotide sequence dataset. See Altschul et al. (1990) J. Mol. Biol., 215:403-10. The BLAST algorithm identifies regions of local similarity and then moves bi-directionally until the BLAST score declines. Another useful tool is BLAT. See Kent W J. BLAT-The BLAST-Like Alignment Tool. Genome Research, April 12(4):656-64. 2002. ProbeSpec is another useful tool that calculates the numbers of mismatches of nearest hits. See Doron Lipson, Peter Web, Zohar Yakhini (2002) “Designing Specific Oligonucleotide Probes for the Entire S. cerevisiae Transcriptome”, WABI '02, 17-21/9/02, Rome.

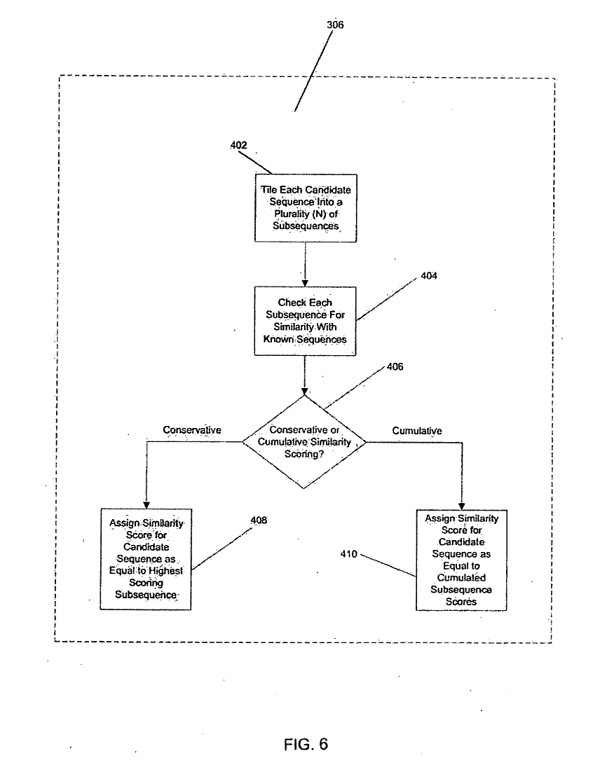

In some embodiments, subsequences of candidate sequences are screened for similarity against known biological sequences of an organism (or organisms) of interest. Referring now to FIG. 6, in some embodiments, a given candidate sequence can be subdivided into a plurality of overlapping or non-overlapping subsequences 402, each of which is then screened for similarity against known biological sequences of an organism of interest 404. For example, a candidate sequence having a length of 60 bases could be subdivided into three distinct subsequences wherein the first subsequence comprises bases 1-30 of the candidate sequence, the second subsequence comprises bases 1545 of the candidate sequence, and the third subsequence comprises bases 30-60 of the candidate sequence. Then each of these subsequences can be compared with a database of known sequences to check for significant similarity 404. It is believed that screening subsequences can offer advantages in that it can make it less likely that any sub-region within a given candidate sequence has a significant match from within the genome or transcriptome of the organism of interest. However, in some embodiments similarity screening is performed using the full candidate sequences.

Similarity can be scored in various ways. In some embodiments, histograms showing the closest matches found are prepared for each sequence or subsequences. Specifically, a histogram is generated showing the number of hits as a function of “distance” of candidate sequences or subsequences from known sequences within the genome or transcriptome of the organism of interest. For example, a distance of 0 base pair(s) corresponds to a candidate sequence that has a direct match in the known sequences within the genome or transcriptome of the organism of interest. Similarly, a distance of 1 base pair(s) corresponds to a candidate sequence having a match in the known sequences within the genome or transcriptome of the organism of interest that is different by only 1 base. Then a score is assigned based on the histogram with “smaller distance” hits (more similar) increasing the score more than “longer distance” hits (less similar). For example, each hit with a distance of 1 base pair might result in increasing the total score for the candidate sequence by 15 units whereas each hit with a distance of 2 base pairs might result in increasing the total score for the candidate sequence by only 12 units. This is only one example of how similarity can be scored. It will be appreciated that scoring can be conducted in many different ways as desired.

In the example of similarity screening performed on subsequences after subdividing the candidate sequences, scoring can be tallied in either a conservative or cumulative manner (see decision 406 in FIG. 6). In some embodiments of the conservative approach 408, scoring can be done by calculating the distribution of similarity scores for each of the subdivided subsequences from a given candidate sequence. Then, the subsequence having the highest similarity score to any sequence from the organism of interest is used to set the score for the overall candidate sequence from which the subsequences are taken. For example, if there are three subsequences in a given candidate sequence and one of the sequences has a score that is higher than the other two, then that higher score is taken as the score for the whole candidate sequence.

Alternatively, similarity scoring for candidate sequences can be done in a cumulative manner. In some embodiments of the cumulative approach 410, the similarity scores for each subsequence are calculated and then cumulated or averaged. For example, assuming there are 3 subsequences for a given candidate sequence and each subsequence produces similarity scores of X, Y, and Z respectively, then the similarity score for the given candidate sequence can be set as either the sum of X, Y, and Z or the average of X, Y, and Z. While some specific examples of calculating similarity scores for candidate sequences have been illustrated herein, it will be appreciated that there are many other ways of calculating similarity scores.

After similarity scores are calculated for candidate sequences, those sequences resulting in scores that indicate significant similarity with one or more naturally occurring sequences in the genome or transcriptome of the organism of interest are removed from the candidate sequence pool. The precise cut-off level for similarity scores will depend on various factors including the length of the candidate sequences, the stringency of wash steps used in the hybridization protocol for the array of interest, scoring method, etc.

Candidate probe sequences that have significant similarity to naturally occurring sequences are undesirable for use as negative controls. In some embodiments, a BLAST raw score (S) is used to select those sequences that do not have significant similarity to known biological sequences. It will be appreciated that BLAST raw score thresholds can be set as desired. In some embodiments, candidate negative control sequences producing any matches against biological sequences with a BLAST raw score of greater than or equal to about 20 are not used. In some embodiments, candidate negative control sequences producing any matches against biological sequences with a BLAST raw score of greater than or equal to about 25 are not used. In some embodiments, candidate negative control sequences producing any matches against biological sequences with a BLAST raw score of greater than or equal to about 30 are not used. In some embodiments, candidate negative control sequences producing any matches against biological sequences with a BLAST raw score of greater than or equal to about 30.23 are not used.

In some embodiments, candidate sequences predicted to form a hybrid with any naturally occurring sequence in the genome or transcriptome of the organism of interest having a predicted Tₘ sufficiently high that the hybrid would be predicted not to melt off during the most stringent post-hybridization was step used in the hybridization protocol are removed from the candidate sequence pool. In some embodiments, candidate sequences having sequence identity of greater than 10 contiguous complementary base pairs, or equally stable longer homologous sequences containing deletions or mismatches, are removed from the candidate sequence pool. In some embodiments, candidate sequences having sequence identity of greater than 15 contiguous complementary base pairs, or equally stable longer homologous sequences containing deletions or mismatches, are removed from the candidate sequence pool.

Closely related to similarity screening, some embodiments can include screening candidate probes for hybridization potential. Hybridization potentials can be calculated using various algorithms known to those of skill in the art. By way of example, hybridization potentials for given sequences can be calculated using a program available online at The Bioinformatics Center at Rensselaer and Wadsworth website (bioinfo.rpi.edu).

One manner of expressing hybridization potential is as ΔG (change in Gibbs free energy) in units of kcals/mol. In some embodiments, candidate sequences having hybridization potential with any naturally occurring biological sequence of a magnitude greater than or equal to −5 kcal/mol are discarded. In some embodiments, candidate sequences having hybridization potential with any naturally occurring biological sequence of a magnitude greater than or equal to −10 kcal/mol are discarded. In some embodiments, candidate sequences having hybridization potential with any naturally occurring biological sequence of a magnitude greater than or equal to −15 kcal/mol are discarded.

In some embodiments, the selected biological probes from the array of interest and/or the pool of candidate probes are screened by their predicted melting temperature with their respective hypothetical complements. In the denaturation of DNA, melting temperature is taken as the midpoint of the helix-to-coil transition. It will be appreciated that there are many different algorithms known to those of skill in the art that allow the prediction of melting temperature based on primary structure (the sequence itself). Examples of such algorithms include that described in Dimitrov and Zuker (2004) Biophysical Journal 87:215-226. The higher the melting temperature, the more energetically stable the duplex or hybridization is.

In some embodiments, candidate sequences having a predicted melting temperature outside the range of about 75° C. to about 85° C., assuming molecule concentrations of between about 1×10⁻⁸ M and 1×10⁻¹⁰ M, are discarded. In some embodiments, candidate sequences having a predicted melting temperature outside the range of about 78° C. to about 82° C., assuming molecule concentrations of between about 1×10⁹ M and 1×10⁻¹⁰ M, are discarded. In some embodiments, candidate sequences having a predicted melting temperature outside the range of about 79.5° C. to about 80.5° C., assuming molecule concentrations of between about 1×10⁹ M and 1×10⁻¹⁰ M, are discarded.

Thermodynamic properties related to the formation of stable structures, such as hairpins, can be calculated in an analogous manner to those of duplex formation. This information can similarly be used to reject candidate sequences if it is likely that the probe will exist in a hairpin formation in solution under the hybridization conditions.

Some embodiments include screening techniques that rely on dataset(s) containing known biological sequences from the organism of interest. Some arrays are designed for use with samples taken from specific organisms. The specific organism(s) that a given array is designed to test samples from is the “organism(s) of interest”. Many projects being conducted by those of skill in the art continue to add to the total pool of known biological sequences for many different organisms. The dataset used for similarity screening can be drawn from one or more databases.

Exemplary databases containing known biological sequences include the NCBI nt database (ncbi.nih.gov), the TIGR (The Institute for Genomic Research) gene indices (tigr.org/tdb/tgi/index.shtml), and the NCBI's Unigene datasets (ncbi.nlm.nih.gov/entrez/query.fcgi?db=unigene). In some embodiments, screening techniques are performed against one or more of the NCBI nt dataset, the TIGR gene indices, and the NCBI's Unigene unique datasets for H. sapiens, A thaliana, and C. elegans.

Those of skill in the art will appreciate that there are also other databases that are available and that contain additional sequences from many different organisms. Publicly available sequence databases include those maintained by: GenBank (Bethesda, Md. USA) (ncbi.nih.gov/genbank/), European Molecular Biology Laboratory's European Bioinformatics Institute (EMBL-Bank in Hinxton, UK) (ebi.ac.uk/embl/), the DNA Data Bank of Japan (Mishima, Japan) (ddbj.nig.ac.jp/), the Ensembl project (ensembl.org/index.html), and The Institute for Genomic Research (TIGR) (tigr.org). Examples of databases that can be obtained and/or searched through the NCBI web portal (ncbi.nih.gov) include Entrez Nucleotides (including data from GenBank, RefSeq, and PDB), all divisions of GenBank, RefSeq (nucleotides), dbEST, dbGSS, dbMHC, dbSNP, dbSTS, TPA, UniSTS, PopSet, UniVec, WGS, Entrez Protein (including data from SwissProt, PIR, PRF, PDB, and translations from annotated coding regions in GenBank and RefSeq), RefSeq (proteins), and many others.

It will be appreciated that some datasets are directed to certain types of sequence information. By way of example, some datasets are directed to genomic sequences, while other datasets are directed to expressed sequences. The appropriate dataset for use will depend on both the type of array intended (e.g., CpG island analysis) and the identity of the organism of interest.

Some embodiments include using a computer system to screen candidate sequences against databases of known sequences. Many available sequence databases can be accessed with computer programs in a way that facilitates automated screening of candidate sequences. Some embodiments include a computer program that automatically screens candidate sequences against databases of known sequences.

Some embodiments include empirically validating candidate sequences. Candidate sequences can be empirically validated by putting the sequences on a test array and then testing hybridization of a sample with sequences on the test array.

In some embodiments, the disclosure provides methods for screening candidate probe sequences, in order to obtain candidates for use as negative control probes, comprising: selecting a subset of probe sequences from a set of sequences randomly; generating a plurality of candidate probe sequences by randomly permuting the selected probe sequence; and screening the candidate probe sequences for sequence similarity to biologically occurring sequences. In some embodiments, the method further comprises selecting a negative probe sequence from the candidate probe sequences wherein the negative probe sequence does not have significant sequence similarity to the biologically occurring sequences.

Probe sequences can additionally be screened based on melting temperature (Tm). In some embodiments, the method comprises discarding candidate sequences having a melting temperate (Tm) outside the range of about 78° C. to about 82° C.

In some embodiments, one or more steps of the method can be performed using a computer.

In some embodiments, the biologically occurring sequences comprise at least 50%, at least 90% or the entire genome of a biological organism, for example, the genome of a mammal such as a human being. In some embodiments, the biologically occurring sequences comprise at least 50%, at least 90% or the entire transcriptome of a biological organism, for example, the transcriptome of a mammal such as a human being. In some embodiments, screening the candidate probe sequences for sequence similarity to biologically occurring sequences comprises screening a set of candidate probe sequences against a database of known sequences. In some embodiments, the set of sequences includes sequences complementary to nucleic acid sequences from an organism of interest, and the database comprises sequences from the organism of interest.

In some embodiments, screening the candidate probe sequences for sequence similarity to biologically occurring sequences comprises subdividing each candidate probe sequence into a plurality of corresponding candidate probe subsequences. The method can further comprise scoring the sequence similarity of each candidate probe sequence according to the sequence similarity of the corresponding candidate probe subsequences.

Methods according to some embodiments of the disclosure can further comprise generating a database of negative probe sequences. As discussed above, in some embodiments, a negative probe sequence does not have significant sequence similarity to biologically occurring sequences, such as for example, the genomic sequences of an organism (e.g., a mammal, such as a human being). In some embodiments, the genomic sequences comprise at least about 50%, at least 90% or 100% of the genomic sequences of an organism, such as a mammal (e.g., a human being). In some embodiments, the biologically occurring sequences comprise the sequences of a transcriptome and in some embodiments, at least 50%, at least 90%, or 100% of the transcriptome of a mammal, such as a human being.

In some embodiments, the methods comprise receiving sequence information for a negative probe sequence and synthesizing the negative probe sequence. Probe sequences can be synthesized by a variety of methods, including, but not limited to in situ synthesis on a solid support (e.g., an array substrate).

The methods can further include empirically testing candidate probe sequences by contacting the probe sequences to a test sample of target sequences and monitoring binding of the probe sequences to the target sequences. For example, candidate probe sequences can be included on an array substrate which can then be contacted with target sequences. The array substrate can additionally include one or more test sequences designed to specifically hybridize to one or more sequences in a biological sample comprising the biologically occurring sequences.

A negative probe sequence can be included in a probe set, which can be immobilized on an array for a hybridization-based assays. For example, the probe sequence can be included on an array used in a methylation assay. Optionally, the probe can be empirically validated as described above before inclusion in the probe set.

In some embodiments, methods according to the disclosure further comprise synthesizing one or more negative control probe sequences. In some embodiments, a negative control probe sequence comprises a sequence length of 10 to 200 bases. In some embodiments, a negative control probe sequence comprises a sequence length of 60 bases. In some embodiments according to the disclosure, a probe includes a negative control sequence and a cleavable site for releasing the negative control probe from an array substrate on which it is immobilized. The probe can additionally or optionally include primer recognition sites for binding to a primer so that the probe can be copied in the presence of a primer, a polymerase and suitable reagents for performing a primer extension and/or amplification reaction.

In some embodiments, the disclosure further provides a probe sequence comprising a negative control probe sequence and a biological probe sequence (i.e., a sequence designed to specifically hybridize to a biologically occurring sequence) for detecting a target sequence in a sample. In some embodiments, the negative control probe sequence is proximal to a solid support on which the probe is immobilized, to link the biological probe sequence to the solid support (either directly or via an additional chemical moiety to which the negative control probe sequence is attached). In some embodiments, an additional parameter used to screen the negative control probe sequence is an absence of secondary structure or ability to form hairpins, such that the negative control probe sequence has minimal likelihood of forming secondary structure. In some embodiments, the negative control probe sequence moves the biological probe sequence off the surface of the microarray and increases hybridization potential of the biological probe sequence (e.g., by reducing steric hindrance and increasing overall sequence accessibility).

In some embodiments, the disclosure provides an array comprising at least one probe comprising a negative control probe sequence and a biological probe sequence. In still some embodiments, the array comprises a plurality of probes comprising a negative control probe sequence and a biological probe sequence. Within the plurality, the negative control probe sequences can be the same or different in some embodiments, though in some embodiments, they are the same. Similarly, within the plurality the biological probe sequence can be the same or different, though in some embodiments, the biological probe sequences are different. In some embodiments, the plurality can comprise the same negative control probe sequences and different biological probe sequences.

In some embodiments, the disclosure also provides a computer readable medium having computer-executable instructions for performing steps of methods as described herein.

In some embodiments, the disclosure provides an apparatus for screening candidate probe sequences, the apparatus comprising: a memory store; and a programmable circuit in electrical communication with the memory store, the programmable circuit programmed to select probe sequences from a set of sequences randomly; generate a plurality of candidate probe sequences by randomly permuting the selected biological probe sequence; and to screen the candidate probe sequences for sequence similarity to biologically occurring sequences. The circuit can be further programmed to select a probe sequence from the candidate probe sequences that does not have significant sequence similarity to the biologically occurring sequences. The programmable circuit can be further programmed to screen candidate probe sequences other properties, such as melting temperature (Tm), for example. In some embodiments, the apparatus further comprises or communicates with a nucleic acid synthesis device, such as an inkjet printer for printing a nucleic acid array. In some embodiments, the nucleic acid synthesis device is responsive to the programmable circuit (e.g., directly or indirectly).

In some embodiments, the disclosure provides a system comprising a database of negative control probe sequences. In some embodiments, sets of negative control probe sequences are selected which correspond to sets of different biologically occurring sequences. A set includes a least one collection of nucleic acid sequences for a biological sample of interest—for example, the set can include human genomic sequences for a biological sample from a human being. In some embodiments, the set includes a plurality of different collections of biologically occurring sequences. For example, a set can comprise mouse genomic sequences and human genomic sequences, such that the database includes a set of negative control probes for a sample of mouse genomic sequences and a set of negative control probes for a sample of human genomic sequences. In some embodiments, the system further comprises a search engine for searching the database in response to an input identifying a set of biologically occurring sequences. For example, in some embodiments, in response to a user request for negative control probes for a sample of human genomic nucleic acids, the search engine will search the database to identify those negative control probe sequences that do not have significant similarity to any human genomic sequences.

In some embodiments, the system communicates with a user device comprising a display for displaying data relating to the negative probe sequences. The data can include but is not limited to: annotation data, sequence data, data relating to empirically determined hybridization properties of the probes, etc. In some embodiments, in response to a selection of one or more negative control probes (e.g., by selecting appropriate areas on a graphical user interface or display), a user can communicate an order for the one or more negative control probes to an entity that can provide the user with such probes (e.g., synthesized on an array or provided in a lyophilized form or in solution).

In some embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Kits

Also provided are kits for use in the subject methods, where in some embodiments such kits can comprise containers, each with one or more of the various reagents utilized in the methods, where such reagents include, but are not limited to, one or more of the following: a control nucleic acid construct as described herein; a nucleic acid vector (e.g., a cloning vector); a restriction endonuclease for use in inserting a double-stranded oligonucleotide into a vector; antibody against 5-methyl-cytosine; antibody against 6-methyl adenosine; antibody against 7-methyl guanosine; a host cell; a host cell transfected with a control nucleic acid construct; a transfection agent; a methylase; a methylation sensitive restriction endonuclease; PCR primers for amplifying a region of a control nucleic acid construct; one or more mixtures of control nucleic acid constructs; one or more mixtures of amlicons of control nucleic acid constructs; labeling reagents, e.g., labeled nucleotides, and the like; a hybridization solution. In some embodiments, reagents can be prepared as a concentrated form (e.g., 10× concentrated) to be diluted upon use.

In some embodiments, a kit can further include instructions for using kit components in the subject methods. The instructions can be printed on a substrate, such as paper or plastic, etc. As such, the instructions can be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc., or can be obtained from the web.

### EXAMPLE 1

### Generation of Negative Control Probes

While it will be appreciated that there are many different techniques for implementing embodiments as program code, this example provides a Matlab script as a specific example. The script takes biological probe sequences and creates random permutations of the sequences to generate a pool of random candidate sequences. The script then subdivides the candidate sequences into subsequences and checks for significant sequence similarity against a table containing known sequences from an organism of interest. The script then creates histograms for similarity scoring purposes.

%MAKENEGATIVECONTROLPROBES (Matlab script)

```
Multiplier=20;
%Biological Probe Sequences:
lod Sequences.mat
for i=1:Multiplier
  %The scramble function randomly permutes the sequences:
  ScrambleSeqs=scramble(Sequences);
    if i==1
      Table60mers.Sequence=ScrambleSeqs;
else
  Table60mers.Sequence=[Table60mers.Sequence;ScrambleSeqs];
        end
end
Table60mers.ProbeID=[1:length(Table60mers.Sequence)]’;
Table60mers.Start=ones(size(Table60mers.ProbeID));
%Tile 30-mer sub-probes through 60-mer probes at15-base intervals:
Table30mers=subdivideprobes(Table60mers,30,15);
Table30mers.ProbeID60mer=Table30mers.ProbeID;
Table30mers.ProbeID=Table30mers.ProbeID*1000+Table30mers.Start;
save WGA2_CandNegCont_Set2_Table30mers.mat Table30mers
save WGA2_CandNegCont_Set2_Table60mers.mat Table60mers
List30.ProbeID=Table30mers.ProbeID;
List30.Sequence=Table30mers.Sequence;
%export a text file that can be used by ProbeSpec for homology search of 30-mer test-
sequences against human genome:
```

table2tabtext(List30,‘WGA2_CandNegCont_Set2_Table30mers.lst’)
%RUN PROBESPEC
% load the resulting homology search file with a histogram of hits at various distances
from 0–9 bases from the original 30-mer sequences:
% load HomologyTable:
load WGA2_CandNegCont_Set2_30mers_MAP.mat
% load Table30mers:
load WGA2_CandNegCont_Set2_Table30mers.mat
% join Table30mers & HomologyTable on ProbeID:
HomologyTable.ProbeID=double(HomologyTable.ProbeID)
NewTable30mers=tablejoin(‘left’,Table30mers,HomologyTable,‘ProbeID’,‘=’, ‘ProbeID’)
load WGA2_CandNegCont_Set2_Table60mers.mat
% combine 30mer probes to make 60mer probes:
% add histogram information for each triplet of 30-mer subsequences:
HomologyTable60mers=combinesubseqhomologies(NewTable30mers,
‘ProbeID60mer’,‘Start’)
NewTable60mers=tablejoin(‘left’,Table60mers,HomologyTable60mers,
‘ProbeID’,‘=’,‘UniFullSeqID’)
% Score homologies for each probe, generate HomLogS2B score:
[HomLogS2B,HomCat,NewTable60mers]=categorizehomology(NewTable60mers,1);

save NC_60mersHomologyTable.mat NewTable60mers

% Keep only those probes with the best homology scores, HomLogS2B.
figure, %plot resulting homology score distribution:
hist(Table.HomLogS2B,[floor(min(Table.HomLogS2B)):ceil(max(Table.HomLogS2B))])

### EXAMPLE 2

### Use of Spiking Reagents

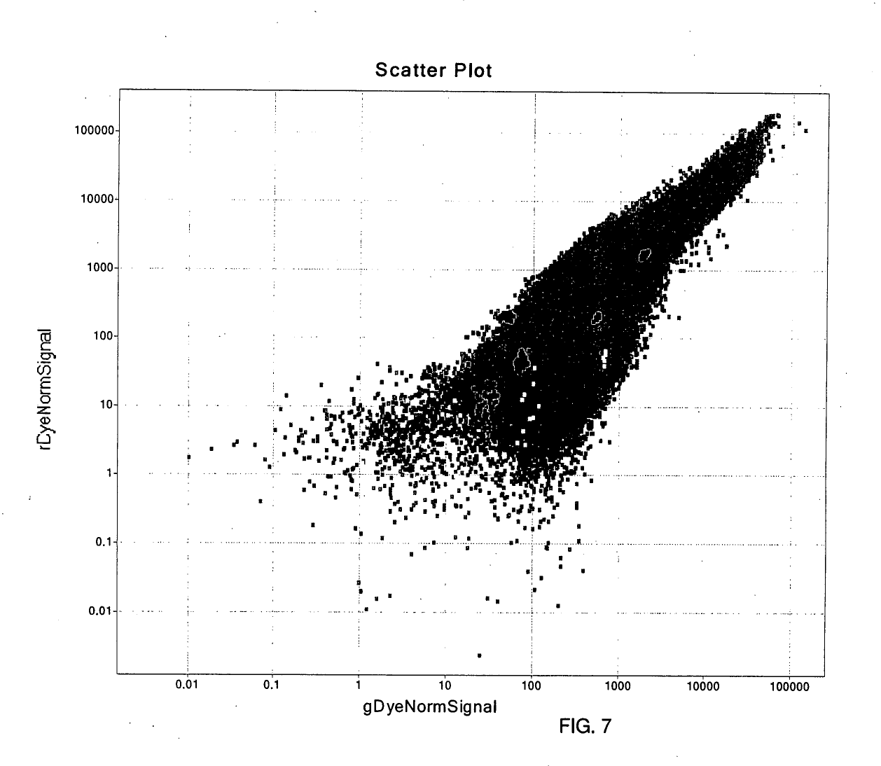

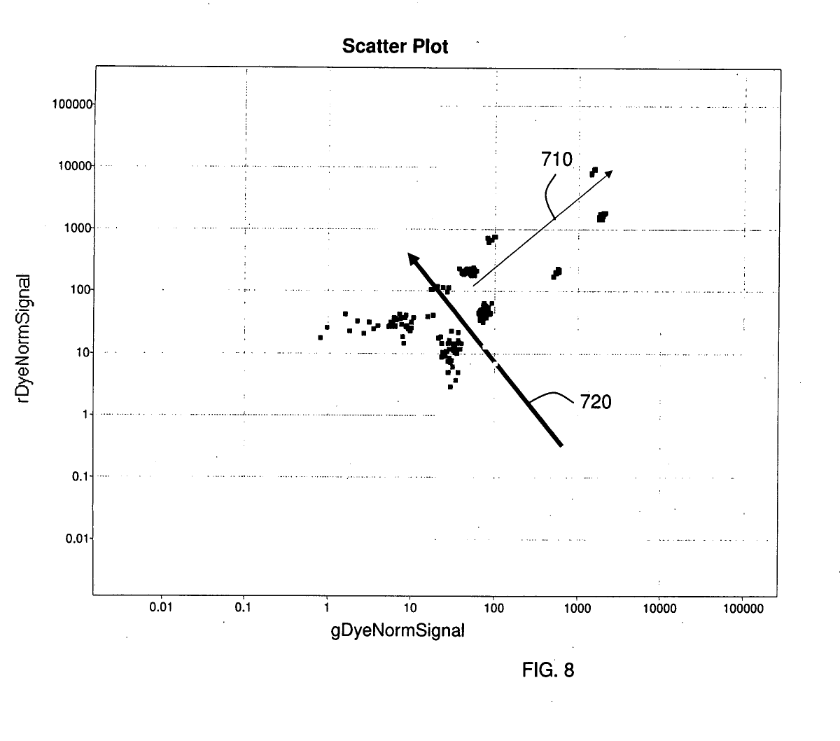

FIGS. 7 and 8 show red and green signal intensities from a representative experiment using a aCpG island array (Agilent catalog no. G4492A) containing amplicons of double-stranded control nucleic acid constructs as spiking reagents as described herein. Each spiking reagent was either unmethlyated, partially or fully methylated in vitro, and added to genomic DNA (human female genomic DNA (Promega catalog no. G1521)) in one of several different concentrations, 5 pg, 50 pg, or 500 pg, to assess linearity of the isolation method. In each experiment, a portion of the genomic DNA/spiking reagent mixture was saved for labeling as the “reference” in the experiment. The remainder of the sample was subjected to a method for isolation of 5-methyl-cytosine DNA using anti-5-methyl cytosine antibody (Eurogentic (Belgum) catalog no. BI-MECY-1000) essentially according to the procedure found in Weber et al. (2006). The isolated DNA was labeled with Cyanine5/red using a conventional labeling protocol (Agilent Array CGH Labeling Kit Plus, catalog no. 5188-5309). The reference channel (Cyanine3/green) was pre-immunoprecipitated DNA (and also contained the spiking reagents).

Fully methylated spiking reagents exhibited the highest ratio of red/green indicating that they were preferentially isolated in the immunoprecipitation procedure (blue points). The partially methylated spiking reagents (red points) exhibited a lower degree of enrichment as their ratio of red/green is lower. The remaining spiking reagents (yellow points) were from the unmethylated spiking reagents, and exhibited no enrichment and a low ratio of red/green. The grey points are the red/green ratios of the genomic probes in the experiment.

A different mixture of spiking reagents (Table 2) was used (containing various different concentrations of spiking reagents in each mixture) for each degree of methylation (unmethylated, partial, full) and containing different genome equivalents: Unmethylated: 3708(10×), 6331(1×), 0984(1×); partially methylated: 0984(1×), 3708(10×), 3499(100×), 6331(1×); fully methylated: 0361(1×), 4040(1×), 2007(10×), 5489(10×), 8976(100×).

In FIG. 8, arrow 710 indicates the trend of increasing signal with increasing copy number. Arrow 720 indicates the trend of higher observed ratio of red/green in fully methylated spiking reagents, and partially methylated spiking reagents.

The spiking reagents listed in Table 2 were prepare by PCR amplification (using SEQ ID NO:45 and SEQ ID NO:46 as PCR primers) of 20 different lambda gt11 constructs each of which contained a unique ˜60 pb insert at the EcorR1 site.

TABLE 2

| Spiking reagent | |
|---|---|
| SEQ ID NOs. of plus and minus strands | Methylation status* |
| 5, 6 | hMe 1 |
| 23, 24 | |
| 1, 2 | Me 10 |
| 43, 44 | Me 1 |
| 33, 34 | hMe 10 |
| 31, 32 | |
| 27, 28 | |
| 39, 40 | unMe 1 |
| 19, 20 | |
| 35, 36 | |
| 29, 30 | |
| 9, 10 | hMe 100 |
| 3, 4 | Me 1 |
| 37, 38 | unMe 10 |
| 7, 8 | unMe 10 |
| 41, 42 | unMe 100 |
| 11, 12 | Me 10 |
| 21, 22 | hMe 1 |
| 15, 16 | unMe 1 |
| 17, 18 | Me 100 |

Key:
Me = fully methylated with SssI methyltransferase (New England Biolabs catalog no. MO226S). The SssI methyltransferase methylates all cytosine residues (C⁵) within the double-stranded dinucleotide recognition sequence 5′ . . . CG . . . 3′.
hMe = partially methylated with HhaI methyltransferase (New England Biolabs catalog no. MO2175). The HhaI methyltransferase modified the internal cytosine residue (C⁵) of the sequence GCGC unMe = unmethylated.
1 = 1 genome equivalent (5 pg) used in the experiment.
10 = 10 genome equivalents (50 pg) used in the experiment.
100 = 100 genome equivalents (500 pg) used in the experiment.

The various embodiments described above are provided by way of illustration only and should not be construed to limit the claims. Those skilled in the art will readily recognize various modifications and changes that can be made without following the example embodiments and applications illustrated and described herein, and without departing from the true spirit and scope of the disclosure or the following claims.

What is claimed is:

1. A control nucleic acid construct comprising:
a double-stranded nucleic acid vector comprising an insert comprising a sequence complementary to a negative control sequence, wherein said insert comprises a methylated methyltransferase recognition site.

2. The control nucleic acid construct of claim 1, wherein the length of said construct is in the range of 2 kilobases to 100 kilobases.

3. The control nucleic acid construct of claim 1 wherein said insert has a sequence length of 10 to 200 bases.

4. The control nucleic acid construct of claim 1 wherein said insert has a sequence length of 60 bases.

5. The control nucleic acid construct of claim 1, wherein said methyltransferase recognition site has been methylated by an in vitro method.

6. The control nucleic acid construct of claim 1, wherein the vector comprises a viral nucleic acid sequence.

7. The control nucleic acid construct of claim 6, wherein the vector comprises lambda phage gt11 and wherein said restriction site comprises an EcoR1 site.

8. The control nucleic acid construct of claim 1, wherein said methyltransferase recognition site comprises a CpG dinucleotide.

9. The control nucleic acid construct of claim 1, wherein said methyltransferase recognition site comprises CpG, CpA, CpT, CpNpG, ApG, GpG, CCGG, GGCC, or TCGA.

10. The control nucleic acid construct of claim 1, wherein said methyltransferase recognition site comprises a methylation site comprising 5-methyl cytidine, 6-methyl adenosine, or 7-methyl guanosine.

11. The control nucleic acid construct of claim 1 comprising lambda gt11 and an insert comprising a sequence selected from the group consisting of: SEQ ID NO:1, SEQ ID NO:2, SEQ ID NO:3, SEQ ID NO:4, and SEQ ID NO:5.

12. The construct of claim 1 wherein said vector has been modified to reduce the number of methyltransferase recognition sites therein.

13. The construct of claim 12 wherein said vector has been modified to reduce the number of CpG dinucleotides therein.

14. The control nucleic acid construct of claim 1, wherein said insert comprises a plurality of methyltransferase methylation sites.

15. The control nucleic acid construct of claim 1, wherein said methyltransferase recognition site is fully methylated.

16. The control nucleic acid construct of claim 1, comprising another insert flanking said insert, said another insert having a length of up to 1000 nucleotides and comprising a methyltransferase recognition site.

17. The control nucleic acid construct of claim 16, wherein said methyltransferase recognition site of said another insert is fully methylated.

18. The control nucleic acid construct of claim 16, wherein said another insert comprises a plurality of methyltransferase recognition sites, and wherein at least some of said plurality of methyltransferase recognition sites are fully methylated.

19. An amplified segment of a nucleic acid having the sequence of the control nucleic acid construct of claim 1, the amplified segment comprising said insert, and wherein the methyltransferase recognition site of said insert is fully methylated.

20. The amplified segment of claim 19 wherein said methyltransferase recognition site comprises a CpG dinucleotide.

21. The control nucleic acid construct of claim 19, wherein the length of said amplified segment is about 2 kilobases.

22. A control nucleic acid construct comprising:
a double-stranded nucleic acid vector comprising a first insert comprising a sequence complementary to a negative control sequence,
a second insert flanking said first insert, said second insert having a length of up to 1000 nucleotides and comprising a methyltransferase recognition site.

23. The construct of claim 22, wherein the sequence of said first insert comprises 10 to 80% methyltransferase recognition sequences.

24. The construct of claim 23, wherein the sequence of said first insert comprises 10 to 80% CpG dinulceotides.

25. The construct of claim 22, wherein the sequence of said second insert comprises 10 to 80% methyltransferase recognition sequences.

26. The control nucleic acid construct of claim 22 comprising a third insert flanking said first, said third insert having a length of up to 1000 nucleotides and comprising a methyltransferase recognition site.

27. The construct of claim 26 wherein said methyltransferase recognition site of said third insert is methylated.

28. The construct of claim 26, wherein the sequence of said third insert comprises 10 to 80% methyltransferase recognition sequences.

29. A composition comprising:
a mixture of a first control nucleic acid construct and a second control nucleic acid construct having the same sequence as said first construct,
wherein said first control nucleic acid construct comprises a nucleic acid vector comprising an insert comprising a sequence complementary to a negative control sequence, wherein said insert comprises a plurality of methyltransferase recognition sites,
wherein in said first control nucleic acid construct, none of the methyltransferase recognition sites are methylated, and
wherein in said second control nucleic acid construct, all of the methyltransferase recognition sites are fully methylated.

30. The composition of claim 29, wherein the ratio of said first control nucleic acid construct to said second control nucleic acid construct is in the range of from 1:100 to 100:1.

31. A composition comprising:
a mixture of a first batch of an amplicon obtained from a control nucleic acid construct and a second batch of said amplicon,
wherein said first control nucleic acid construct comprises a nucleic acid vector comprising an insert comprising a sequence complementary to a negative control sequence, wherein said insert comprises a plurality of methyltransferase recognition sites, wherein said amplicon comprises said insert,
wherein in said first batch, none of the methyltransferase recognition sites are methylated, and
wherein in said second batch, all of the methyltransferase recognition sites are fully methylated.

32. The composition of claim 31, wherein the ratio of said first batch to said second batch is in the range of from 1:100 to 100:1.

33. The construct of claim 31 wherein at least some of said methyltransferase recognition sites comprise CpG dinulceotides.

34. A single-stranded spiking reagent, comprising:
a sequence complementary to a negative control sequence, wherein said sequence comprises at least one methylated base.

35. The single-stranded spiking reagent of claim 34, comprising:
a second sequence contiguous with said first sequence, wherein said second sequence comprises at least one methylated base.

36. The single-stranded spiking reagent of claim 35 wherein said second sequence comprises a sequence that is not substantially complementary to nucleic acids expected to be in a sample under investigation.

37. A method of preparing a nucleic acid for use as a spiking reagent, the method comprising:
providing a control nucleic acid construct comprising: a nucleic acid vector comprising an insert comprising a sequence complementary to a negative control sequence, wherein said insert comprises a methyltransferase recognition site, and
methylating said methyltransferase recognition site.

38. The method of claim 37, wherein said methylating is by an in vitro process.

39. A method for use in assessing the methylation status of a sample of double-stranded nucleic acid, the method comprising:
a) adding a control nucleic acid construct to said sample, said construct comprising a nucleic acid vector comprising an insert comprising a sequence complementary to a negative control sequence, wherein said insert comprises a methylation site,
b) enriching said sample for nucleic acids comprising a methylated methylation site, and
c) detecting nucleic acids obtained in step (b) to assess the methylation status of said sample.

40. The method of claim 39 wherein said methylation site comprises 5-methyl cytidine.

41. The method of claim 39, further comprising a step of fragmenting said nucleic acid of said sample prior to said enriching.

42. The method of claim 39 wherein said enriching comprises immunoprecipitating nucleic acids comprising a methylated methylation site.

43. The method of claim 39, further comprising before step (a): separating the strands of double-stranded nucleic acid fragments in the sample.

44. The method of claim 39 comprising an amplification step prior to step (b).

45. The method of claim 39 comprising a labeling step prior to step (b).

46. The method of claim 39 wherein said detecting comprises microarray analysis.

47. The method of claim 39 further comprising:
(d) detecting nucleic acids obtained in step (a) by microarray analysis.

48. A method for detection of changes in nucleic acid methylation in a patient over time comprising: (i) obtaining a tissue specimen from the patient at a time point; (ii) repeating step (i) for at least one further time point; (iii) extracting nucleic acid from each tissue specimen to provide a sample of nucleic acid for each time point, and (iv) carrying out the method of claim 39 on each nucleic acid sample for each time point to characterize whether, and/or to what extent, the nucleic acid sequence is methylated.

49. A method for preparing a control nucleic acid construct comprising the steps of:
a) providing a cloning vector,
b) inserting into said vector a control nucleic acid molecule having a sequence complementary to a negative control sequence,
c) transferring the product of step (b) into competent cells, and growing said cells,
d) obtaining a control nucleic acid construct from said cells, said construct comprising said vector with said control nucleic acid molecule inserted therein, and
e) methylating all methylation sites in the control nucleic acid construct of step (d).

50. A kit for performing methylation analysis of a nucleic acid sample, said kit comprising:
a control nucleic acid construct comprising a vector
said vector comprising an insert comprising a sequence complementary to a negative control sequence, said insert comprising a methyltransferase recognition site,
means for methylating said methyltransferase recognition site.

51. The kit of claim 50 wherein said methyltransferase recognition site comprises CpG dinucleotide.

52. The kit of claim 50 further comprising amplification primers for amplifying a segment of said construct, said segment comprising said insert.

53. The kit of claim 50 further comprising instructions for using the kit in a methylation detection assay.

54. The kit of claim 53 wherein said instructions comprise instructions for using the kit in a microarray hybridization assay.

55. The kit of claim 50 wherein the control nucleic acid construct comprises an isolated nucleic acid molecule comprising lambda gt11 and an insert comprising a sequence selected from the group consisting of: SEQ ID NO:1, SEQ ID NO:2, SEQ ID NO:3, SEQ ID NO:4, and SEQ ID NO:5.

56. The kit of claim 50, wherein said kit comprises means for enriching a sample for methylated nucleic acids.

57. The kit of claim 56, wherein said means for enriching comprises an antibody.

58. A kit for performing methylation analysis of a nucleic acid sample, said kit comprising:
a nucleic acid comprising an first sequence complementary to a negative control sequence, said first sequence comprising a methylated nucleoside.

59. The kit of claim 58 further comprising instructions for using the kit in a methylation detection assay.

60. A kit for performing methylation analysis of a nucleic acid sample, said kit comprising:
a single-stranded spiking reagent, comprising:
a sequence complementary to a negative control sequence, wherein said sequence comprises at least one methylated base, and
instructions for using the kit in a microarray hybridization assay.

61. A kit for performing methylation analysis of a nucleic acid sample, said kit comprising:
a single-stranded spiking reagent, comprising:
a first sequence complementary to a negative control sequence, and a second sequence contiguous with said first sequence, wherein said second sequence comprises at least one methylated base, and
instructions for using the kit in a microarray hybridization assay.

62. A kit comprising:
an amplicon obtained from a control nucleic acid construct,
wherein said control nucleic acid construct comprises a nucleic acid vector comprising an insert comprising a sequence complementary to a negative control sequence, wherein said insert comprises at least one methyltransferase recognition site,
wherein said amplicon comprises said insert, and
instructions for using the kit in a methylation detection assay.

63. A kit comprising:
a first batch of an amplicon obtained from a control nucleic acid construct and a second batch of said amplicon,
wherein said control nucleic acid construct comprises a nucleic acid vector comprising an insert comprising a sequence complementary to a negative control sequence, wherein said insert comprises at least one methyltransferase recognition site,
wherein said amplicon comprises said insert,
wherein in said first batch, none of the at least one methyltransferase recognition site is methylated,
wherein in said second batch, the at least one methyltransferase site is methylated, and
instructions for using the kit in a methylation detection assay.
